(19) United States
(12) Patent Application Publication
(10) Pub. No.: US 2013/0246086 A1
(43) Pub. Date: Sep. 19, 2013

(54) HEALTH QUANT DATA MODELER

(71) Applicants: Johnathan C. Mun, Pleasanton, CA (US); Thomas Michael Schmidt, Iowa City, IA (US)

(72) Inventors: Johnathan C. Mun, Pleasanton, CA (US); Thomas Michael Schmidt, Iowa City, IA (US)

(21) Appl. No.: 13/786,786

(22) Filed: Mar. 6, 2013

Related U.S. Application Data
(60) Provisional application No. 61/612,941, filed on Mar. 19, 2012.

(51) Int. Cl. G06Q 50/22

(52) U.S. Cl. CPC G06Q 50/22; G06Q 10/067
USPC 705/2

(57) ABSTRACT

Embodiments of the present invention are applicable in the field of finance, health care, employee benefits, math, and business statistics and are originated to provide real health-care decision analysis, risk analysis, and option analytics to corporate entities and individual participants, the need for which has arisen from the passage of the Patient Protection and Affordable Care Act. The present Health Quant Data Modeler (HQDM) invention is designed as an application that integrates optimization, cohort analysis, forecasting, real options, and risk-based Monte Carlo simulation into a comprehensive utility for corporations as well as individuals in order to make better health-care related decisions.

### CROSS REFERENCE TO RELATED APPLICATIONS

This application claims the benefit of U.S. Provisional Patent Application No. 61/612,941 filed Mar. 19, 2012, the entire disclosure of which is incorporated herein by reference.

### FIELD OF THE INVENTION

The present invention is applicable in the field of finance, health care, employee benefits, math, and business statistics and was originated to provide real health-care decision analysis, risk analysis, and option analytics to corporate entities and individual participants, the need for which has arisen from the passage of the Patient Protection and Affordable Care Act. The present Health Quant Data Modeler (HQDM) invention is designed as an application that integrates optimization, cohort analysis, forecasting, real options, and risk-based Monte Carlo simulation into a comprehensive utility to assist corporations as well as individuals in making better health-care related decisions.

### COPYRIGHT AND TRADEMARK NOTICE

A portion of the disclosure of this patent document contains materials subject to copyright and trademark protection. The copyright and trademark owner has no objection to the facsimile reproduction by anyone of the patent document or the patent disclosure, as it appears in the U.S. Patent and Trademark Office patent files or records, but otherwise reserves all copyrights whatsoever.

### BACKGROUND OF THE INVENTION

The following provides the background and context for this invention.

The history and growth of employer-sponsored health insurance in this country changed precipitously after World War II when health insurance enrollment grew from 21 million in 1940 to 143 million in 1950 directly as a result of government intervention by excluding this form of employee compensation from federal taxation. On Mar. 23, 2010, U.S. President Barack Obama signed into law the Patient Protection and Affordable Care Act as amended by the Health Care and Education Reconciliation Act of 2010, known as the Affordable Care Act (ACA), as a another form of government intervention that sets forth health-care policy changes intended to further expand insurance coverage in the United States. This legislation is set to transform the health-care and health-care financing systems for the next generation, with some of the implications noted here for corporations, employers, individuals, insurance companies, and the individual states within the United States.

Employers may either terminate their employer sponsorship of health insurance coverage and pay an annual penalty tax or provide health insurance coverage to their employees according to newly mandated requirements per the ACA. For example, employers must expand eligibility to all full-time employees, offer a prescribed benefits package with a minimum actuarial benefit plan value, prohibit lifetime restrictions on annual benefit maximums, and limit employee contributions. Individuals are mandated to either purchase coverage or pay an annual penalty tax for noncompliance. Insurance companies are subject to medical loss ratio requirements that require rebates on excess profits generated when the claims loss ratios are below certain thresholds. State governments have to either build a state-based Health Insurance Exchange or allow the federal government to step in and provide those Health Insurance Exchange services.

The U.S. Census Bureau provides the following information with respect to the number of people and the number of firms in the market that are important market-sizing determinants in the development of this invention (Source: U.S. Census Bureau, American Fact Finder, Economic Data Set, 2010 American Community Survey, 1 Year Estimates).
In 2010, there were 309 million people living in the United States; 265 million under the age of 65 and 191 million between 18 and 64. There were 150 million people (Source: Kaiser/HRET, Employer Health Benefits, 2011 Annual Survey, pg. 42) covered by private health insurance, 57% (Source: Kaiser/HRET, The Uninsured, A Primer, Key Facts About Americans Without Health Insurance, December 2010, pg. 2) of whom were covered through an employer-sponsored benefit. Two-thirds of these individuals had coverage provided to them by firms with 200 or more employees (Source: Kaiser/HRET, Employer Health Benefits, 2011 Annual Survey, pg. 8). Finally, 41 million people were reported to have no health insurance (Source: Kaiser/HRET, The Uninsured, A Primer, Key Facts About Americans Without Health Insurance, December 2010, pg. 2).
As of 2008, there were 5.9 million firms (Source: U.S. Census Bureau Statistics, Employment Size of Employer and Non-Employer Firms, 2008, Table 2a) with approximately 121 million employees on payroll. Small employers (defined as fewer than 100 employees) constituted 98% of the firms and 35% of the employees. Large employers (defined as greater than or equal to 100 employees) constituted 2% of the firms and 65% of the employees. A further stratification of the large employer segment shows that employers with 500 or more employees represented 0.3% of the firms and half of the total number of employees, and employers with 10,000 or more employees represented 0.1% of the employers and 27% of the employees.

The following descriptions represent the implications to each group.
Large Employers. Employers with fifty (50) or more full-time employees are considered large employers for purposes of this legislation and must either provide health insurance coverage (“play”) or realize a penalty (“pay”). To “play” means that the employer must comply with the mandates for expansion of eligibility, provide a specified level of benefits, accept the prohibition on limits, and restrict employee contributions and cost sharing within mandated limits.
Small Employers. A qualified small employer is one with fewer than twenty-five (25) employees. Such small employers will have the option of offering coverage to their employees within the newly created Health Insurance Exchange under a small-business health options program. The small employer will elect to provide coverage as a group within the Exchange and will specify the level of coverage (i.e., 60% [bronze], 70% [silver], 80% [gold], or 90% [platinum] level) within which the employees will have the opportunity to enroll with any of the insurance carriers that provide coverage at this level within the Exchange.
Individuals. Employees are confronted with an individual mandate. Individuals will have the option of retaining coverage with their employer, purchasing coverage through an Exchange (the only place where a premium tax credit and a cost-sharing subsidy are available), purchasing coverage in the private insurance market, or paying a noncompliance penalty.

As corporate entities attempt to strategically manage their health-care expenditures, they are faced with assessing their health-care reform options. For example:
Abandonment Option. Will an employer decide that the nondeductible tax penalty and lost tax shields compared to their cost of offering or continuing to offer coverage is preferred and therefore elect to abandon employer-provided health insurance coverage?
Expansion Option. Is the attraction of new employees and the retention of existing employees the most significant argument for an employer to adopt, continue, or expand contributions or eligibility in the sponsorship of health insurance coverage?
Switching Option. The Health Insurance Exchanges, by legislative design, are intended for individuals and small employers, and yet large employers are considering the option of using them for their part-time employee and retiree populations. They are considering the feasibility of terminating their health plans and shifting 100% of their employees to the Exchanges for coverage. Is this a viable option for a large employer in spite of the fact these Exchanges are designed for individual and small group coverage and, if so, will there be a large-employer domino effect?
Stage-Gate Option. A typical employer offers its employees some choices, e.g., a standard option, high option, and a high deductible health plan option. According to the Office of Personnel Management, the federal government offers 207 different plan choices, but, as a practical matter, the choice for a federal employee is between six and fifteen plan options (Source: Federal Employees Health Benefits Program: Available Health Insurance Options, CRS, RS21974, Nov. 18, 2010). Will employers with employees dispersed throughout the country determine that the Exchange constitutes more flexibility and plan options than their current health plan offerings? Will they elect to separate their plans by retaining employer-sponsored health insurance coverage for corporate and terminating coverage for a division in order to provide Exchange access?
Contraction Option. Defined contribution health accounts have been used for retiree health benefits for many years now. Retirees are credited with a fixed-dollar amount for each year of plan participation or some variation of age plus years of service. The main advantage of establishing these types of accounts has been the predictability for the employers as they fund specific dollar amounts. The retirees assume the liability for the difference between the actual amount of coverage purchased in the market and the specific dollar amount funded by their employers. How will employers assess whether to move to such a defined contribution approach for their active population? Will they be able to preserve premium tax and cost-sharing subsidies in the process?

There are many additional issues that influence these options and will ultimately influence the decision about which option the employer will ultimately adopt. For example:
Premium Tax Credits. Premium tax credits and cost-sharing subsidies are based on an individual employee's income and the number of his or her dependents. Employers with higher salaries may determine that there will be minimal, if any, of these tax credits or subsidies for their employee population. However, employers with lower salaries may determine that there will be significant premium tax credits and subsidies available for their employee population. How will an employer go about making this assessment as to whether its population will benefit from the premium tax credits, Medicaid, and cost-sharing subsidies?
Self-Insurance Concerns. What recourse does an employer have when the plan can no longer place a dollar limit on an annual or lifetime basis for essential health benefit coverage and stop loss coverage may not be available or too expensive? Will an employer simply decide to self-fund until a point where it is economically feasible to terminate its self-funded plan and migrate to the Exchange because the Exchange must accept the employer as a risk without regard to its historical claims experience? What happens if the stop loss carrier non-renews at year end after reimbursement of a catastrophic claim?
Enrollment and Communication. Will an employer who currently has annual open enrollment meetings, sponsors benefit fairs, and provides communication materials decide that this function may be shifted to the Exchange?
Anti-Selection. Economic self-interest creates openings for an employer to break with groupthink. Employers are currently structuring contributions and models to migrate high-cost users into fixed-cost arrangements and low-cost users into variable-cost arrangements. This will play itself out into an analysis of an experience-rated versus a community-rated result. Will the employer anti-select such that it will use the Exchange for its retirees and part-time employees or find a way to shift high-cost users to the Exchange through organizational structures that will not be discriminatory?

The Office of the Actuary for the Centers for Medicare & Medicaid Services annually produces projections of health-care spending and in 2010 reported total national health-care expenditures of $2.6 trillion (Source: Office of the Actuary, Centers for Medicare & Medicaid Services, NHE Projections 2010-2020, Forecast Summary and Selected Tables, Table 1), with $822.3 billion for private health insurance. In 2014, it projects 13.9 million people will enroll in Health Insurance Exchange plans in their first year and that private health insurance enrollment will peak at 195 million in 2015, but decline thereafter.

The Congressional Budget Office report estimated that only 9 to 10 million people (or about 7% of employees) who are currently covered by employer-sponsored health insurance would switch to subsidized Exchange policies in 2014.

McKinsey & Company published conclusions as to the impact of health-care reform on employer options. Its conclusions were based on a survey of more than 1,300 employers across industries, geographies, and employer sizes, as well as other proprietary research (Source: McKinsey Quarterly; “How U.S. health care reform will affect employee benefits,” McKinsey & Company, June 2011). It reported that 30% of employers and 28% of large employers said they will definitely or probably stop offering employer-sponsored insurance in the years after 2014. Among employers with a high awareness of reform (50% and above), most will pursue some alternative to traditional employer-sponsored insurance with those alternatives including dropping coverage, offering it through a defined-contribution model, or, in effect, offering it only to certain employees. McKinsey & Company concluded that many employers will be shifting from employer-sponsored insurance, and it would be unlikely that only one company in an industry or geography will move away from it, producing a domino effect throughout the country.

There is an expectation that the market will be dramatically altered by the passage of this legislation. It is a fact that there is little health insurance competition in many states where one insurer claims half of the individual and small-group fully insured market. What is less discussed is the national predominance of the Blue Cross Blue Shield Association, United Health Group, CIGNA, and Aetna coverage of over half of the covered population in the United States. It has been the history and practice of the insurance business to be built around relationship-based and transactional placements. Insurance carriers have built wholesale distribution channels around this high level of fragmentation. The advent of ACA's medical loss ratio requirement has now placed significant pressure on these insurers. The current distribution channel is set to fracture under the weight of the medical loss ratio requirements and heightened executive-level pressure on human resources departments to provide more value and analysis.

There will be a far greater level of disruption in the market than estimated by either the Office of the Actuary for CMS or the Congressional Budget Office, a position more closely aligned with McKinsey's conclusions.

The related art is represented by the following references of interest.

U.S. Pat. No. 8,095,392 (application Ser. No. 11/336,070 filed on Jan. 20, 2006) by Owen, Daniel L. (Los Altos, Calif.) (herein, “Owen”), describes a computer-implemented method for the execution of a risk-management application for performing decision logic that presents alternatives relating to a risk exposure of a family of a user; the risk management application selected by the user from a plurality of different risk-management applications each capable of performing different decision logic and using different databases; retrieving first information from a database in accordance with the decision logic; and processing at least a portion of the first information in order to generate second information not originally included in the database. It should be noted that such asset risk-management is defined to include management of any assets, cash flow, budgets, etc. that are affected by risk-mitigation instruments (e.g., health insurance, automobile insurance, life insurance, financial investments, long-term care insurance, home security devices, vehicle security devices, insurance, investments, etc.). Owen merely describes a design that helps to determine whether personal risks should be retained or transferred. Owen does not suggest any method of how to analyze employer-sponsored health insurance offerings, how to integrate individual and corporate health-care data into the running of forecasts and Monte Carlo risk simulations, or how to develop a plurality of strategic real options on making health-care insurance decisions. The Owen invention is strictly on the application of general risk management where data is collected and collated using computer-based logic to filter the data and perform relational database management tasks.

U.S. Pat. No. 8,090,562 (application Ser. No. 12/425,956 filed on Apr. 17, 2009) by Snider, James V. (Pleasanton, Calif.); Heyman, Eugene R. (Montgomery, Md.) (herein, “Snider”), describes a clinical evaluation for determination of disease severity and risk of major adverse cardiac events (MACE), e.g., mortality due to heart failure. It is useful for the prognostic evaluation of subjects, in particular for the prediction of adverse clinical outcomes, e.g., mortality, transplantation, and heart failure. Snider only applies to a clinical predictive modeling application through the use of biomarkers. Snider does not suggest how employer-sponsored health insurance offerings and their respective plan designs should be changed to improve outcomes. Snider strictly concerns the application of clinical management of key biomarkers to impact significant cardiac events, and that is not what this current invention is about.

U.S. Pat. No. 8,041,580 (application Ser. No. 12/039,131 filed on Feb. 28, 2008) by Sholtis, Steven (El Dorado Hills, Calif.) (herein, “Sholtis”), et al. describes a computer system-implemented method and process for forecasting the consequences of health-care utilization choices whereby health data associated with a user is obtained and analyzed to determine disease risk factors. The health-care utilization consequences report can include health-care recommendations, economic information, actuarial information, and comparisons between implementing/not implementing the health-care recommendations. Sholtis includes data representing information related to any historical and/or present user illnesses; data representing information related to any historical and/or present user injuries; data representing information related to any historical and/or present preventative health care received by the user; data representing information related to any historical and/or present medications taken by the user; data representing information related to any historical and/or present illness associated with the user's family and/or the user's family health history; data representing information related to any historical and/or present user residences; data representing information related to any historical and/or present user occupations; data representing information related to any historical and/or present user environmental exposures that could affect the user's predisposition to a particular type of disease; and/or data representing any other information related to the user's historical state of health or current state of health, or that is determined of value in projecting the user's future state of health. Sholtis describes a personal economic forecast of health-care consumption based on genetics, actual utilization history, demographic data, and personal family history. Sholtis is strictly for the application of personal health risk assessment.

U.S. Pat. No. 8,005,690 (application Ser. No. 11/835,593 filed on Aug. 8, 2007) by Brown, Stephen J. (Woodside, Calif.) (herein, “Brown”), describes the modeling and scoring risk-assessment and a set of insurance products derived therefrom. Risk indicators are determined at a selected time. A population is assessed at that time and afterward for those risk indicators and for consequences associated therewith. Population members are coupled to client devices for determining risk indicators and consequences. A server receives data from each client and, in response thereto and in conjunction with an expert operator, (1) reassesses weights assigned to the risk indicators, (2) determines new risk indicators, (3) determines new measures for determining risk indicators and consequences, and (4) presents treatment options to each population member. The server determines, in response to the data from each client, and possibly other data, a measure of risk for each indicated consequence or for a set of such consequences. The expert operator uses this measure to determine either (1) an individual course of treatment, (2) a resource utilization review model, (3) a risk-assessment model, or (4) an insurance pricing model for each individual population member or for selected population subsets. Brown is a health-risk-assessment tool that captures medical, psychological, and lifestyle questions along with biometric data to develop some form of a risk scoring application. Brown is strictly an application to assess personal risk factors that may be used to assess current courses of treatments and potentially be integrated into an insurance pricing model by loading premiums for higher risk factors.

U.S. Pat. No. 8,000,977 (application Ser. No. 10/799,042 filed on Mar. 11, 2004) by Achan, Pradeep Padmakshan (Castro Valley, Calif.) (herein, “Achan”), describes a method and system for development of health-care information systems (HIS). The method includes providing software programming interfaces for development of application modules, communication interfaces for establishing communication between various modules, and resource management interfaces for allocation of resources such as memory. Achan discloses a design for clinical information exchange among nurses, doctors, and the ward by capturing and sharing biometric, drug interaction, and drug and diet interaction information. Achan is strictly for the application of clinical information exchange.

U.S. Pat. No. 7,958,002 (application Ser. No. 12/607,838 filed on Oct. 28, 2009) by Bost, James (Washington, D.C.) (herein, “Bost”), describes a system and method for measuring the relative economic benefits from services offered by health-care plans. Bost is strictly for the measurement of NCQA health plans using productivity metrics.

U.S. Pat. No. 7,912,739 (application Ser. No. 10/691,762 filed on Oct. 23, 2003) by Colley, John Lawrence (Richmond, Va.), et al. (herein, “Colley”), describes a method for managing health plans and includes the use of theoretically derived mathematical models. The Colley method may be used in the analysis of health insurance products. The Colley method may also assist in the selection of a particular health plan's benefit and contribution strategy. The analysis may further be used in the selection of a health plan's funding arrangement. The system and methods described in Colley do not have the breadth and depth of trending and forecasting techniques for self-funded plan analysis; do not have a contribution optimization utility that both initially calculates an effective employer target percentage and can subsequently perform a reverse calculation of phantom rates based on a revised user update of an effective employer target percentage; have limited application to normal distribution versus a best fit among thirty or more distribution types; have adopted an inferior approach by not using a per member per month (PMPM) calculation methodology; and have mostly adopted subjective index valuations on benefit modeling valuation calculations that function as single point estimates of future plan value versus Monte Carlo risk simulations on plan costs based on various input assumptions. Colley is a benefits modeling application with single point comparative estimates on funding types and contribution structures.

U.S. Pat. No. 7,912,734 (application Ser. No. 11/679,267 filed on Feb. 27, 2007) by Kil, David H. (Santa Clara, Calif.) (herein, “Kil”), describes apparatuses, computer media, and methods for supporting the health needs of a consumer by processing input data. An integrated health management platform supports the management of health care by obtaining multidimensional input data for a consumer, determining a health-trajectory predictor from the multidimensional input data, identifying a target of opportunity for the consumer in accordance with the health-trajectory predictor, and offering the target of opportunity for the consumer. A health benefit plan is offered from a set of health benefit plan configurations. Responses are received from a questionnaire to members of a consumer group, and preferred health benefit plans chosen by members of the group are predicted. From the responses, an overall enrollment distribution is estimated. A plurality of health benefit plans is offered to the group when a minimum economic objective is obtained from the set of health benefit plan configurations. Kil is a predictive modeling risk scoring application that uses claims data, self-reported data, consumer behavior marketing data, disease clustering, and disease progression probabilities as part of a methodology to develop health plan offerings by integrating trajectory valuations with consumer-preference and projected utility functions.

U.S. Pat. No. 7,813,937 (application Ser. No. 10/360,858 filed on Feb. 6, 2003) by Pathria, Anu K. (San Diego, Calif.), et al. (herein, “Pathria”), describes a transaction-based behavioral profiling, whereby the entity to be profiled is represented by a stream of transactions, which is required in a variety of data mining and predictive modeling applications. An approach is described for assessing inconsistency in the activity of an entity, as a way of detecting fraud and abuse, using service-code information available on each transaction. Pathria describes a fraud and detection health-care provider profiling application.

U.S. Pat. No. 7,769,600 (application Ser. No. 11/933,098 filed on Oct. 31, 2007) by Iliff, Edwin C. (La Jolla, Calif.) (herein, “Iliff”), describes a system and method for allowing a patient to access an automated process for managing a specified health problem called a disease. The system of Iliff performs disease management in a fully automated manner, using periodic interactive dialogs with the patient to obtain health state measurements from the patient, to evaluate and assess the progress of the patient's disease, to review and adjust therapy to optimal levels, and to give the patient medical advice for administering treatment and handling symptom flare-ups and acute episodes of the disease. Iliff describes a clinically based, personalized disease-management application to assist the individual in the long-term management of his or her disease. Iliff does not suggest a method of how to evaluate the effectiveness of population-based disease management programs.

U.S. Pat. No. 7,653,557 (application Ser. No. 11/315,054 filed on Dec. 22, 2005) by Sweetser, Christine B. (Linn Haven, Fla.) (herein, “Sweetser”), describes an advanced primary nurse care system, and a process is disclosed that is client-driven for processing a number of clients in a timely manner with enhanced health-care outcomes. The system and process of Sweetser are sized to provide an optimum patient flow and health care. The system and process of Sweester include a computer network having a central system computer. A computer program resides on the system computer for creating a real-time client record as the client proceeds through the system and process. There is a client station connected in the computer network where the client record is initially created and accessed on subsequent visits using a unique client ID code. Sweetser is strictly a health-care operational application.

U.S. Pat. No. 7,555,438 (application Ser. No. 11/491,035 filed on Jul. 21, 2006) by Binns, Gregory S. (Lake Forest, Ill.); Blumberg, Mark Stuart (Oakland, Calif.) (herein, “Binns”), describes a method of model development for use in underwriting group life insurance for a policy period. The system and method of Binns includes collecting medical claims data for the group to be underwritten, where each medical claim is related to a particular employee of the group. Morbidity categories are provided that categorize the medical claims in the medical claims data. A conditional probability model is developed and applied to the morbidity categories for each employee in the group using his or her medical claims, thereby calculating the expected conditional probability for each employee dying during the policy period. For each employee, an estimate of the expected life claim cost is estimated using an index of the life coverage to salary. Combining the expected conditional probability for each employee dying during the policy period with the estimate of the expected claim cost of death gives an estimate of the group's total life exposure. The system and methods of Binns use medical claims in the underwriting of mortality and morbidity of group life insurance experience.

U.S. Pat. No. 7,493,264 (application Ser. No. 10/166,298 filed on Jun. 11, 2002) by Kelly, Miriam A. (Ridgewood, N.J.); Lotvin, Alan M. (Maple Grove, Minn.) (herein, “Kelly”), describes an interactive computer-assisted method that compiles comprehensive health-care information on patients in a central repository, assesses and analyzes this information, and identifies high utilizers of health-care services through use of a computer and a user associated therewith. The methods of Kelly include the step of creating a central repository of various databases containing patient information, including demographic information and behavior, and, optionally, the results of a core survey of health status questions. Kelly optionally involves the step of determining the appropriate core questions and the criteria to determine whether and when to ask certain questions of particular patients based on their response to prior questions. In summary, Kelly is a combination of a health-risk-assessment questionnaire and a basis for a predictive modeling application.

U.S. Pat. No. 7,392,201 (application Ser. No. 09/861,379 filed on May 18, 2001) by Binns, Gregory S. (Wilmette, Ill.); Blumberg, Mark Stuart (Oakland, Calif.) (herein, “Binns-2”), describes a computer-implemented process of developing a person-level cost model for forecasting future costs attributable to claims from members of a book of business, where person-level data are available for a substantial portion of the members of the book of business for an actual underwriting period, and the forecast of interest for a policy period is disclosed. Binns-2 pertains to health, disability, and life insurance systems, particularly including processing data (in the business of health insurance) for estimating future costs or liability and setting optimal pricing.

U.S. Pat. No. 7,213,009 (application Ser. No. 10/658,998 filed on Sep. 9, 2003) by Pestotnik, Stanley L. (Sandy, Utah), et al. (herein, “Pestrotnik”), describes a method for delivering decision-supported patient data to a clinician to aid the clinician with the diagnosis and treatment of a medical condition. The method of Pestrotnik includes presenting a patient with questions generated by a decision-support module and gathering patient data indicative of the responses to the questions. Each question presented to the patient is based on the prior questions presented and the patient data gathered from the patient. On receiving the patient data from the client module, the patient data is evaluated at the module to generate decision-supported patient data. This supported patient data includes medical condition diagnoses, pertinent medical parameters for the medical condition, and medical care recommendations for the medical condition. At the client module or a clinician's client module, this patient data is presented to the clinician in either a standardized format associated with a progress note or a format selected by the clinician. Pestrotnik describes a method for delivering decision-supported patient data to a clinician to aid the clinician with the diagnosis and treatment of a medical condition.

U.S. Pat. No. 6,381,576 (application Ser. No. 09/212,521 filed on Dec. 16, 1998) by Gilbert, Edward Howard (Plano, Tex.) (herein, “Gilbert”), describes a diagnostic and treatment information data structure that encapsulates, with or without identifying a specific patient, information regarding a particular diagnosis-treatment cycle for an individual patient. In Gilbert, the diagnostic and treatment information data structures for a number of diagnosis-treatment cycles may be combined within a database for analysis in outcomes or cost-effectiveness studies. A relational database that assists the health-care provider in formulating the diagnostic and treatment information data structure for a specific diagnosis-treatment cycle can, within a user interface, display information determined during the outcomes or cost-effectiveness studies to influence the health-care provider at the point of decision. Effective analyses of diagnostic, treatment, and outcomes information and guidance for health-care professionals based on such analyses are thus facilitated. An Internet/intranet database program employing the diagnostic and treatment information data structure contains both clinical and financial information permitting effective filtering and analysis of CPT codes as to accuracy and appropriateness. Gilbert merely describes an operational health-care provider clinical application that facilitates a diagnostic and treatment cycle.

U.S. Pat. No. 6,370,511 (application Ser. No. 09/188,986 filed on Nov. 9, 1998) by Dang, Dennis K. (Phoenix, Ariz.) (herein, “Dang”), describes a computer-implemented method for profiling medical claims to assist health-care managers in determining the cost efficiency and service quality of health-care providers. The Dang method allows an objective means for measuring and quantifying health-care services. An episode treatment group (ETG) is a patient classification unit that defines groups that are clinically homogenous (similar cause of illness and treatment) and statistically stable. The ETG methodology uses service or segment-level claim data as input data and assigns each service to the appropriate episode. The program identifies concurrent and recurrent episodes, flags records, creates new groupings, shifts groupings for changed conditions, selects the most recent claims, resets windows, makes a determination if the provider is an independent lab, and continues to collect information until an absence of treatment is detected. Dang merely describes an application associated with the development of early evidence-based medicine guidelines through the creation of episode treatment groups.

Therefore, there is need in the art for a computer-implemented system and method for providing analysis that corporate entities can use to assess their current arrangements and determine these corporate entities future “play” or “pay” positions. These and other features and advantages of the present invention will be explained and will become obvious to one skilled in the art through the summary of the invention that follows.

### SUMMARY OF THE INVENTION

Accordingly, embodiments of the present invention are configured to preempt this result by promoting the analysis that will lead to a more rapid adoption of alternatives to employer-sponsored health insurance. Preferred embodiments of the present invention extract data from numerous and distinct data elements in order to provide an objective and strategic risk-based real options decision analysis.

The present invention, with its preferred embodiment encapsulated within a Health Quant Data Modeler (HQDM) software, is applicable for the types of analyses that corporate entities need to assess their current arrangements and determine their future “play” or “pay” positions, and Health Quant Individual Modeler (HQIM) software is applicable for the types of analyses that individuals will need to assess the options available to them in the market. In certain embodiments, a HQDM is both a standalone and server-based set of software modules and advanced analytical tools that are used in an innovative way that links various databases and data sources to integrate optimization, cohort, forecasting calculations, real options, and simulation. In other embodiments, the HQDM could be configured to operate as a completely standalone set of software modules, as a server-based set of software modules, or any combination thereof. One of ordinary skill in the art would appreciate that there are numerous configurations that could be utilized in conjunction with a HQDM, and embodiments of the present invention are contemplated for use with any configuration.

According to an embodiment of the present invention, a HQDM may be used for all of its components (i.e., optimization, cohort analysis, forecasting, real options, and Monte Carlo risk simulation) or for its real options component alone. The HQIM may be configured as (i) a standalone set of software modules, (ii) a server-based set of software modules, (iii) an advanced analytical tool set that is used integrate optimization, real options modeling, and simulation, or (iv) any combination thereof. In certain embodiments, an HQIM may be attached to an HQDM as an option for the employees of a population or as a detached capability.

### BRIEF DESCRIPTION OF THE DRAWING

FIG. 1 illustrates a schematic overview of a computing device, in accordance with an embodiment of the present invention.

FIG. 2 illustrates a network schematic of a system, in accordance with an embodiment of the present invention.

FIG. 3 illustrates the three layers: business logic, data access, and presentation.

FIG. 4 and FIG. 5 illustrate the optimization process map.

FIG. 6 and FIG. 7 illustrate the cohort process map.

FIG. 8 and FIG. 9 illustrate the forecasting process map.

FIG. 10 and FIG. 11 illustrate the simulation process map.

FIG. 12 and FIG. 13 illustrate the real options process map.

FIG. 14 illustrates the individual modeler process map.

FIG. 15 illustrates the system's various data input methods in linking existing data tables and databases, using manual inputs, providing the capabilities of computing and creating new data variables, setting simulation assumptions, model fitting existing data to various mathematical and statistical distributions, and SQL data filtering.

FIG. 16 illustrates the system's interconnectivity capabilities and mapping/linking approaches to various database systems such as Excel, SQL Server, Healthcare Data Warehouses, and Internal HRIS systems, as well as other data types and models.

FIG. 17 illustrates the manual data input capabilities of manually entering required input data or uploading data files into the system.

FIG. 18 illustrates the system's data computation process of using existing data variables or numerical inputs to generate new variables.

FIG. 19 illustrates the system's process of setting up various statistical distributions of an input variable for running simulations.

FIG. 20 illustrates the system's process of data model fitting of multiple data points to various statistical distributions of an input variable for running simulations.

FIG. 21 illustrates the SQL data filtering method of generating new variables.

FIG. 22 and FIG. 23 illustrate the repository of data within the data tables.

FIG. 24 illustrates the variable mapping of user data to the required variables in HQDM.

FIG. 25 and FIG. 26 illustrate the process map of the HQDM Model.

FIG. 27 illustrates the charts generated from the analysis.

FIG. 28 illustrates the Reports Dashboard where multiple charts and data tables can be presented in a comprehensive view.

FIG. 29 illustrates the Report Creator where the user can decide which reports to generate.

### DETAILED DESCRIPTION OF THE INVENTION

According to an embodiment of the present invention, the computer-implemented system and methods herein described may be configured to utilize one or more sets of models and algorithms. A preferred embodiment of the present invention has the ability to perform Monte Carlo risk-based simulations, forecasting, fit of existing data, optimization to allocate employer- and employee-based contributions, and linking from and exporting to existing databases and data files.

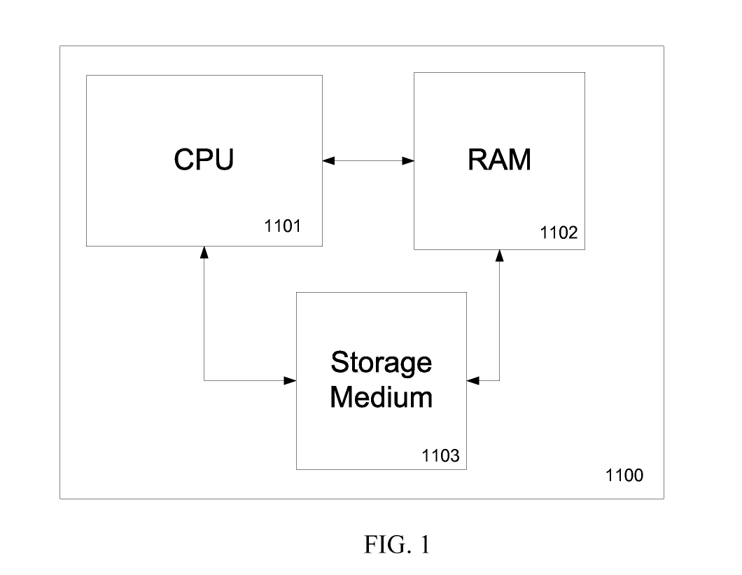

According to an embodiment of the present invention, the system and method are accomplished through the use of one or more computing devices. As shown in FIG. 1, one of ordinary skill in the art would appreciate that a computing device 1100 appropriate for use with embodiments of the present application may generally comprise one or more central processing unit (CPU) 1101, random access memory (RAM) 1102, and a storage medium (e.g., hard disk drive, solid state drive, flash memory, cloud storage) 1103. Examples of computing devices usable with embodiments of the present invention include, but are not limited to, personal computers, smart phones, laptops, mobile computing devices, tablet PCs, and servers. The term “computing device” may also describe two or more computing devices communicatively linked in a manner as to distribute and share one or more resources, such as clustered computing devices and server banks/farms. One of ordinary skill in the art would understand that any number of computing devices could be used, and embodiments of the present invention are contemplated for use with any computing device.

In an exemplary embodiment according to the present invention, data may be provided to the system, stored by the system, and provided by the system to users of the system across local area networks (LANs, e.g., office networks, home networks) or wide area networks (WANs, e.g., the Internet). In accordance with the previous embodiment, the system may comprise numerous servers communicatively connected across one or more LANs and/or WANs. One of ordinary skill in the art would appreciate that there are numerous manners in which the system could be configured, and embodiments of the present invention are contemplated for use with any configuration.

In general, the system and methods provided herein may be consumed by a user of a computing device whether connected to a network or not. According to an embodiment of the present invention, some of the applications of the present invention may not be accessible when not connected to a network; however, a user may be able to compose data offline that will be consumed by the system when the user is later connected to a network.

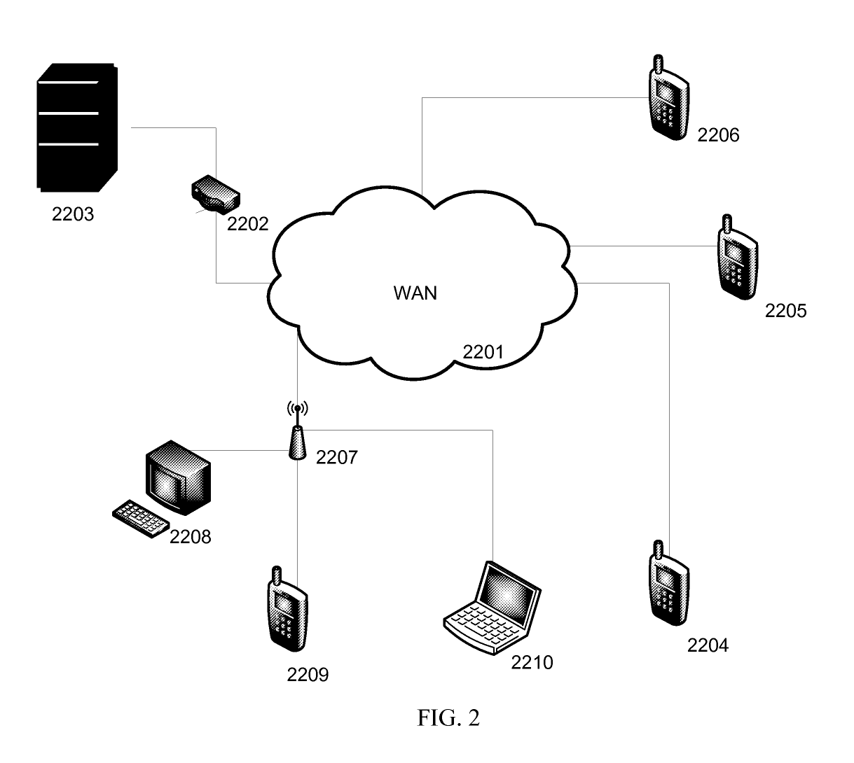

Referring to FIG. 2, a schematic overview of a system in accordance with an embodiment of the present invention is shown. The system consists of one or more application servers 2203 for electronically storing information used by the system. Applications in the application server 2203 may retrieve and manipulate information in storage devices and exchange information through a WAN 2201 (e.g., the Internet). Applications in a server 2203 may also be used to manipulate information stored remotely and process and analyze data stored remotely across a WAN 2201 (e.g., the Internet).

According to an exemplary embodiment, as shown in FIG. 2, exchange of information through the WAN 2201 or other network may occur through one or more high-speed connections. In some cases, high-speed connections may be over-the-air (OTA), passed through networked systems, directly connected to one or more WANs 2201, or directed through one or more routers 2202. Router(s) 2202 are completely optional, and other embodiments in accordance with the present invention may or may not utilize one or more routers 2202. One of ordinary skill in the art would appreciate that there are numerous ways a server 2203 may connect to WAN 2201 for the exchange of information, and embodiments of the present invention are contemplated for use with any method for connecting to networks for the purpose of exchanging information. Further, while this application refers to high-speed connections, embodiments of the present invention may be utilized with connections of any speed.

Components of the system may connect to a server 2203 via WAN 2201 or other network in numerous ways. For instance, a component may connect to the system (i) through a computing device 2212 directly connected to the WAN 2201; (ii) through a computing device 2205, 2206 connected to the WAN 2201 through a routing device 2204; (iii) through a computing device 2208, 2209, 2210 connected to a wireless access point 2207; or (iv) through a computing device 2211 via a wireless connection (e.g., CDMA, GMS, 3G, 4G) to the WAN 2201. One of ordinary skill in the art would appreciate that there are numerous ways that a component may connect to a server 2203 via WAN 2201 or other network, and embodiments of the present invention are contemplated for use with any method for connecting to a server 2203 via WAN 2201 or other network. Furthermore, a server 2203 could be a personal computing device, such as a smartphone, acting as a host for other computing devices to connect to.

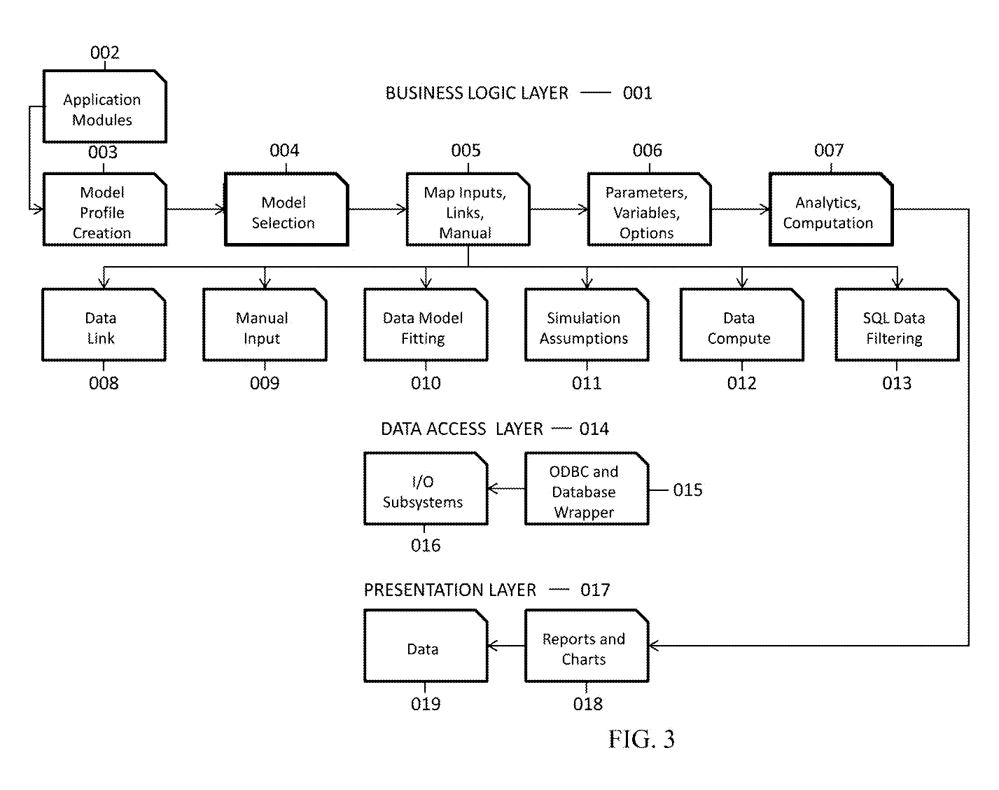

FIG. 3 illustrates the underlying infrastructure of an embodiment of the present invention. In this preferred embodiment, three layers are utilized: a business logic layer, a data access layer, and a presentation layer. The business logic layer 001 contains the application modules 002, that is, the mathematical and financial models, where the user first creates a profile 003 that stores all the input assumptions and then selects the relevant model 004 to run. When the model is selected, the system automatically requests that the required input parameters be mapped 005. Several methods exist for the user at this point to decide where the input data comes from—through some existing data files or database 008; manual inputs through directly typing data into the software 009; using existing data to fit into mathematical distributions through statistical fitting routines 010; or without the use of existing data—to set Monte Carlo simulation parameter assumptions 011, a combination of these approaches through a data compute module 012 by modifying existing variables or through creation of a new variable through the use of SQL expressions 013. One of ordinary skill in the art would appreciate that there are numerous methods for receiving input data, and embodiments of the present invention are contemplated for use with any method for receiving input data.

According to an embodiment of the present invention, if databases or data tables or data files are used and linked in the business logic layer 001, then the method accesses the data access layer 014 through calling a proprietary database wrapper 015 and input-output (I/O) subsystems 016. On completing the variable mapping step 005, the user then sets up the parameters and enters the variables or selects the options 006. Then the analytics and computations occur 007, developing the presentation layer 017 that generates the relevant charts and statistics 018 and allows the computed results to be extracted as flat text files or data tables back into the database 019 as new variables.

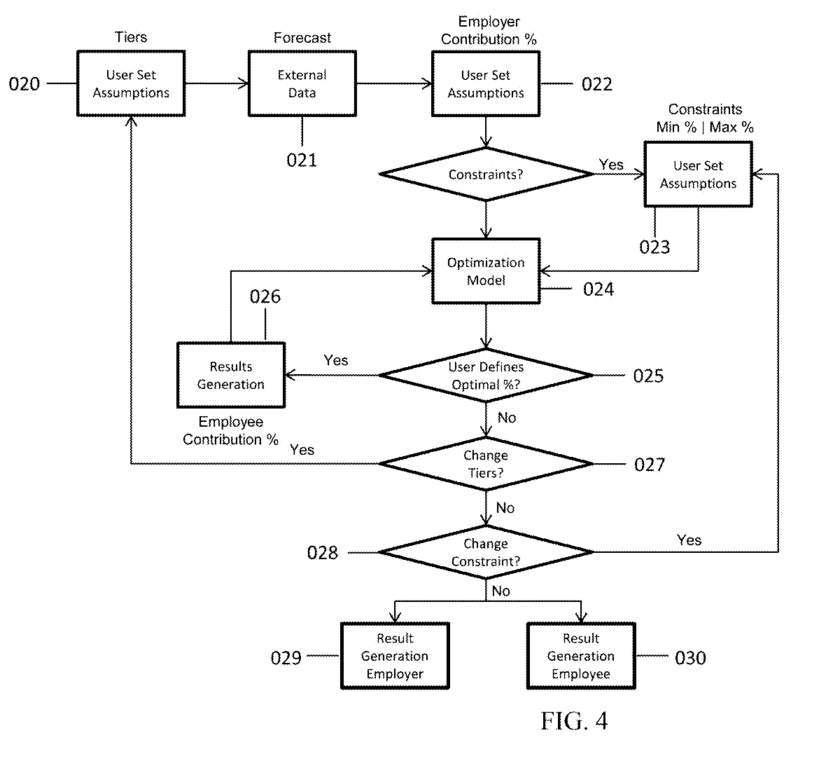

FIG. 4 illustrates the optimization process, in accordance with an embodiment of the present invention, through a flowchart. This process begins with the selection of the tier variable 020 from a drop-down list, which then populates the tier variable column with the appropriate tiers and drop-down lists of each tier to select for prioritization beginning with the first tier and ending with the last tier. The tier variable selected is limited only by the number of tiers constructed within the data tables. A tier is defined as a type or group of similar characteristics (e.g., single person, married person, married with one child, married with two children, and so forth). The optimization amount is entered 021 and, as noted, the value entered is forecasted future period health-care claims. Each tier is then populated with an appropriate allocation ratio 022 by the user (e.g., “employee and spouse” may be populated with a “2” reflecting the assumption that an employee and spouse would be expected to incur twice as many claims as a single employee). Further, a percentage contribution is entered by the user 022 for each tier level based on the modeling objective toward the cost of coverage. For example, a contribution calculation design would be an employer contributing 100% for employee coverage and 50% for dependent coverage. By industry convention this is defined as “noncontributory” for the employee-only portion of the coverage and “contributory” for the dependent portion of the coverage. This means the employee makes no contributions toward the cost of his or her employee-only portion of coverage, but would be required to contribute 50% toward the cost for the additional premium to cover dependents. A minimum and maximum percentage constraint may be placed within each tier by entering the minimum and maximum thresholds 023. This does not have to be the case during the initial calculation to determine the effective contribution percentage, but would be the case (minimum and maximum percentages are optional) during a subsequent optimization run when a target effective employer contribution percentage is targeted. The optimization process is initiated 024 and the target employer effective percentage output calculation is reported 029 as a target effective employer contribution percentage. This calculation is derived from the aggregate dollar amount the employer contributes toward each tier of coverage (the numerator is based on the employer contribution input assumptions) divided by the total employer and employee dollar contributions toward the cost of coverage (the denominator) where such total equals 100% of the initial input 021. After this initial calculation, it is possible to manually enter a different target employer effective percentage 025 and retain, remove, or amend the existing constraints 028. The optimization process is then run 026, a recalculation is executed, and new outputs are produced 029. At this point, the choice exists to change tiers 027, which then initiates the process 020 to recalculate, or change constraints 023, and go through a recalculated optimization process 024 with the results being generated for the employer 029 and the employee 030.

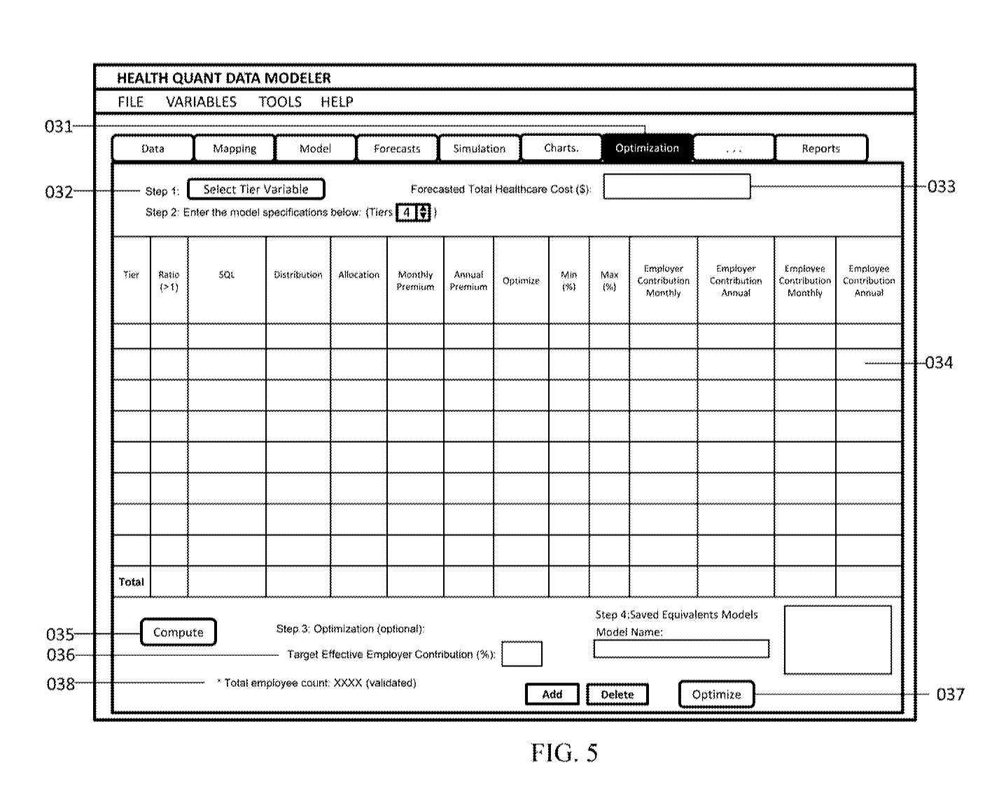

FIG. 5 further illustrates the optimization process, in accordance with an embodiment of the present invention, through a user interface. This process is initiated by selecting the optimization tab 031 and choosing the tier variable 032. The forecasted total health-care cost amount is entered 033. The variables noted in FIG. 4 ranging from user entry of tiers, ratios, contribution percentages, and constraints along with utility generated calculations are entered or calculated 034. The compute function 035 generates the target effective employer contribution percentage 036. The opportunity to change this target effective employer contribution percentage 036 and rerun the optimization 037 is the power of the utility in that a complete recalculation of the results 034 populates the data matrix. Reconciliations are performed within the utility with each calculation, with a total count of employees 038 used as a validation test.

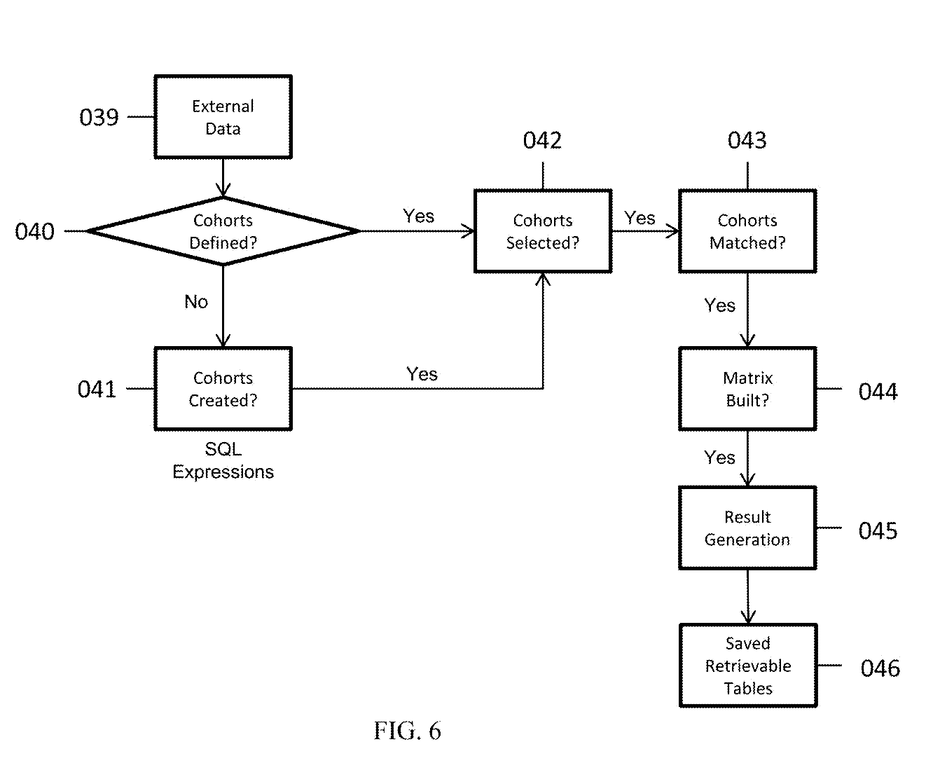

FIG. 6 illustrates the cohort process, in accordance with an embodiment of the present invention, through a flowchart. This process begins by strategically defining the who, what, where, when, why, and how of what needs to be evaluated and then selects from among the available data tables 039. The cohort creation 040 begins by identifying and drawing from among the variables within a specific data table. The building of SQL queries is the single most important component in the ultimate development of the cohorts for measurement 041. These SQL queries allow for great flexibility and are restricted only by the data within the tables themselves. The cohort is then selected 042 and matched 043. The compute function generates the results that supply both the matrix 044 and the graph 045. Each table may be saved and retrieved 046. These tables are exportable into either an Excel worksheet or a text file. In other embodiments of the present invention, the tables may be exported into one or more other file formats. One of ordinary skill in the art would appreciate that there are numerous file formats that could be utilized with embodiments of the present invention, and embodiments of the present invention are contemplated for use with any file format.

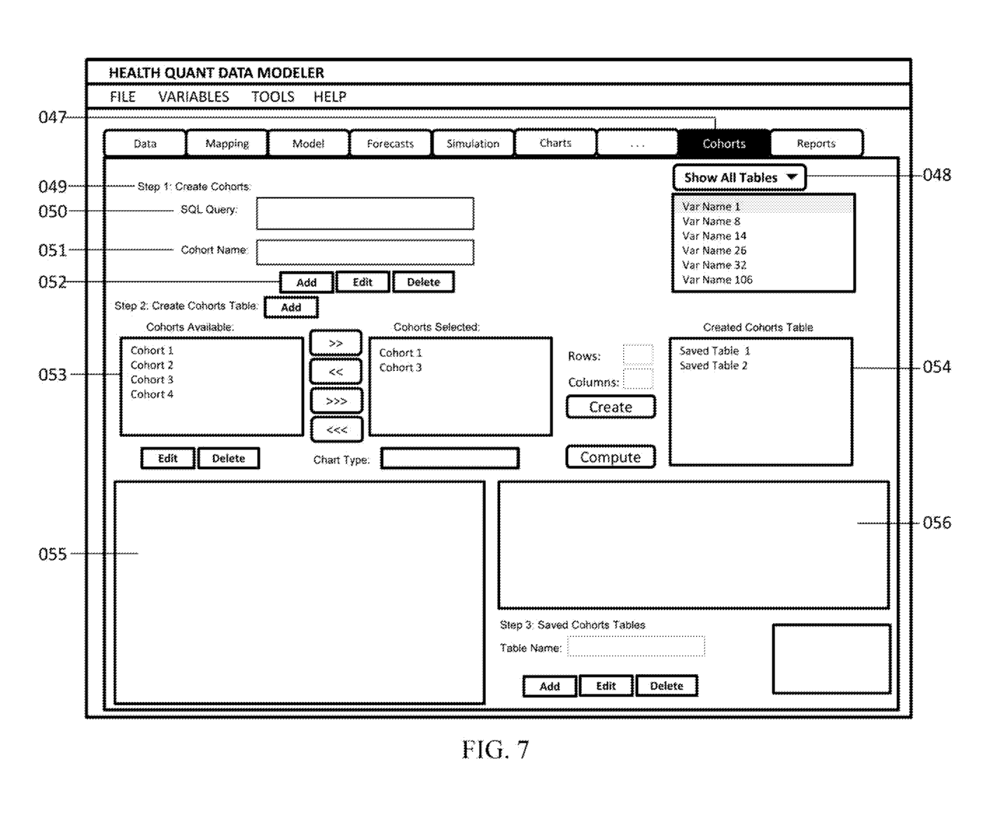

FIG. 7 further illustrates the cohort process, in accordance with an embodiment of the present invention, through a user interface. The process begins in the cohorts tab 047 by first strategically defining the who, what, where, when, why, and how of what needs to be evaluated. The cohort creation begins by identifying and drawing from among the variables within a specific data table 048. It begins with the creation of an SQL query 049 where the expressions are defined by the user 050. The cohort is named 051 and may be amended (added, edited, or deleted) 052. The cohorts noted as available 053 are then selected 054 for the creation of the cohort matrix 054. There is both a graphical representation 055 of the data computation as well as the data calculations within the data computation table 056.

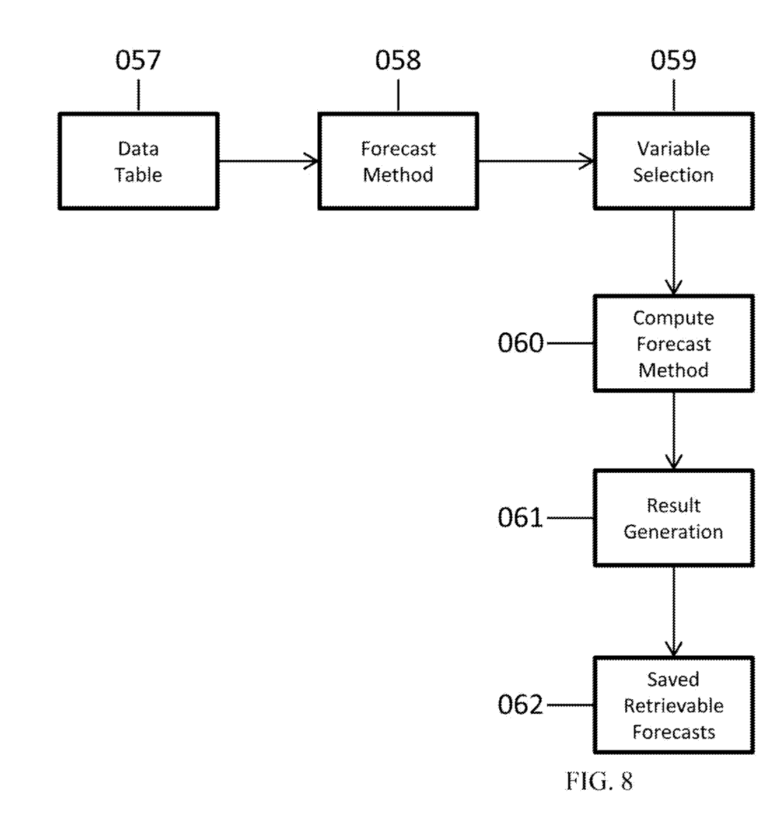

FIG. 8 illustrates the forecasting process, in accordance with an embodiment of the present invention, through a flowchart. This process begins with the decision as to what to forecast and the selection of the data tables available for forecasting 057. The forecast methods are selected 058 from a drop-down list that may include, but is not limited to, such processes as auto-regressive integrated moving average (ARIMA), basic and auto econometrics, time-series analyses (e.g., double exponential smoothing, double moving average, Holt Winters additive, Holt Winters multiplicative, seasonal additive, seasonal multiplicative, single exponential smoothing, single moving average) and trend lines (i.e., exponential, linear, logarithmic, moving average, polynomial, power). One of ordinary skill in the art would appreciate that there are numerous forecast methods that could be utilized with embodiments of the present invention, and embodiments of the present invention are contemplated for use with any forecast method. Each forecast method has its own specific data requirements for entry 059 such as the variables for forecast, seasonality, number of forecast periods, independent variables, dependent variables, and so on. The computation 060 generates a chart, data, and statistics 061. Each forecast may be saved and retrieved 062 at a later time.

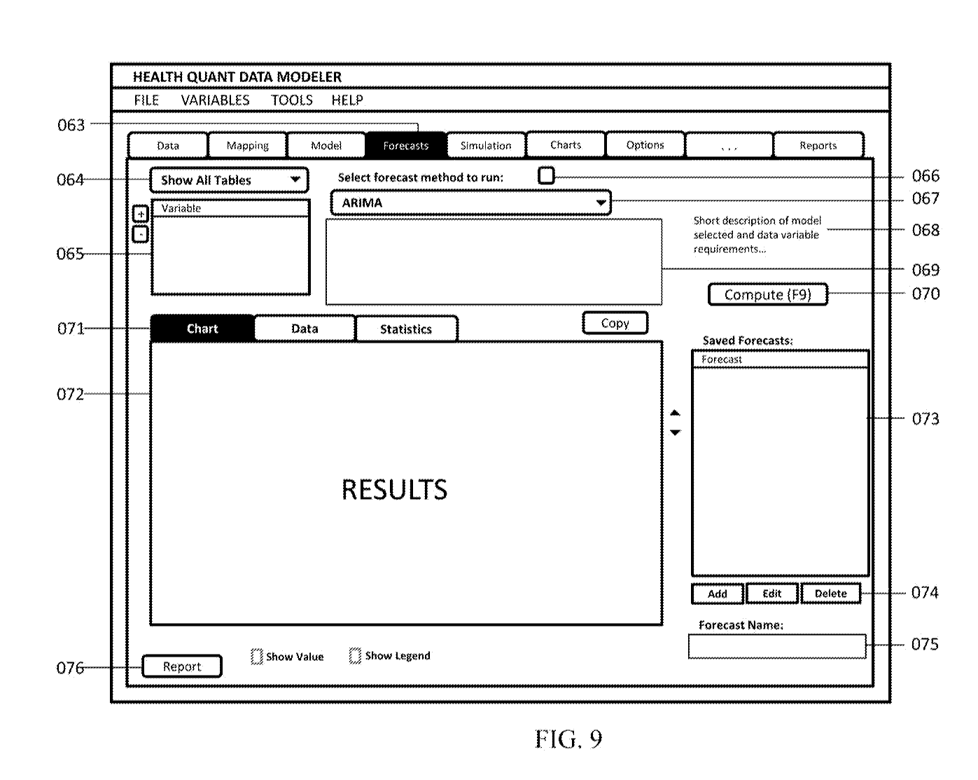

FIG. 9 further illustrates the forecasting process, in accordance with an embodiment of the present invention, through a user interface. This process begins with selecting the tab to forecast 063. Next a choice is made as to the location of the data tables 064 for the variables 065 for inclusion in the forecast. The complete list of forecast methods may be viewed 066 and specific forecast methods may be selected to be shown in the drop-down list 067. There is a short description for each model 068. Each forecast method has its own specific data requirements for entry 069 such as the variables for forecast, seasonality, number of forecast periods, independent variables, dependent variables, and so on. Computation is initiated 070. Charts, data, and statistics are generated 071, with both graphical and tabular results of the data shown 072. Forecasts are named 075 and new forecast models can be added or existing forecast models can be edited or deleted 074. Models are also available for retrieval 073 at a later date. Reports are available for generation and viewing 076 once models have been generated.

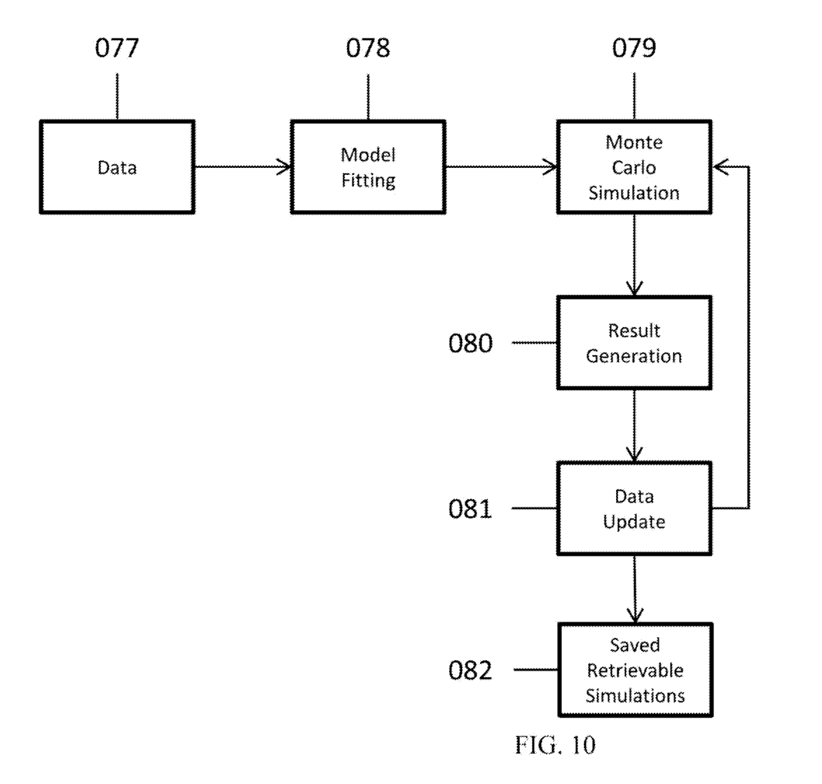

FIG. 10 illustrates the simulation process, in accordance with an embodiment of the present invention, through a flowchart. A significant component to this utility is the use of Monte Carlo risk-based simulation. The Monte Carlo risk-based simulation process is a critical decision support analytic that contributes to improved decision making by migrating from single point estimates to an applied simulation that provides greater levels of confidence from the generated forecasts produced from randomly generated results created from detailed input assumptions and appropriately selected distribution models. It begins with selecting the data for simulation 077, goes through a model fitting exercise 078 by setting the necessary input assumption properties (e.g., distributions [normal, uniform, triangular, etc.] are tested one at a time, the appropriate input parameters for each distribution that best fits the user's historical data is found through optimization, and the resulting best-fitting distribution is selected), and the resulting probability distribution is then generated 079. Results are then simulated using the best-fitting distribution 080. Data may be updated 081 and another simulation run with new results generated 080. The simulations may be saved and retrieved 082 at a later date.

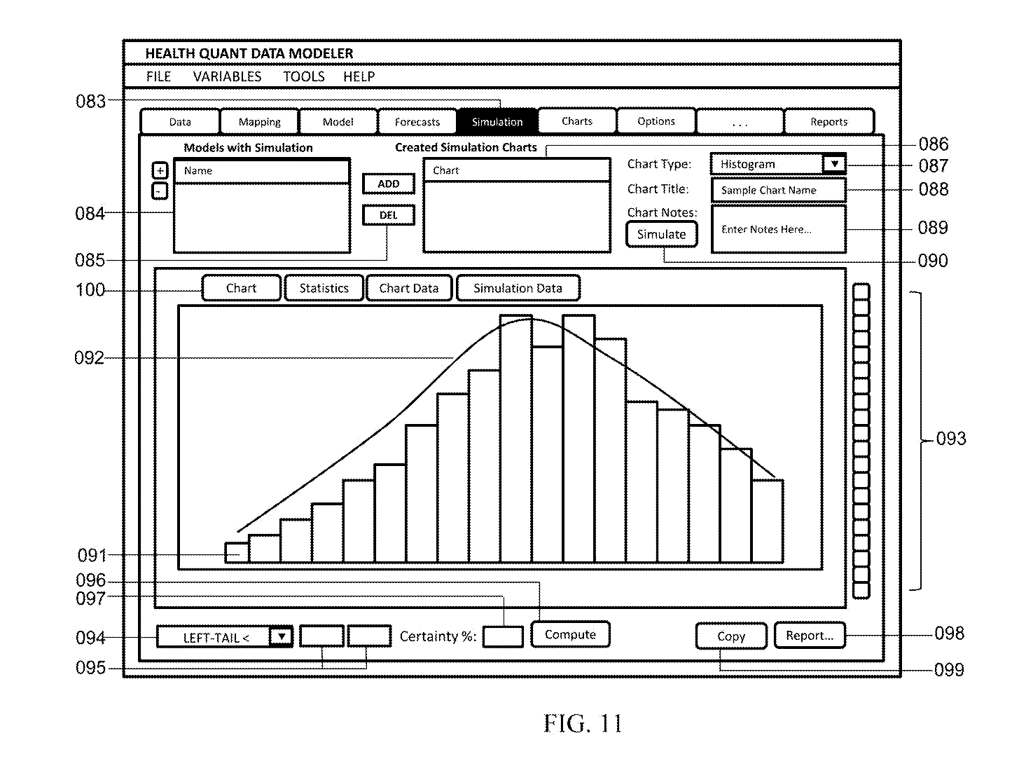

FIG. 11 further illustrates the simulation process, in accordance with an embodiment of the present invention, through a user interface. A Monte Carlo risk-based simulation 083 begins with selecting the data for simulation or selecting an existing simulation 084 that may be added and/or deleted 085. Chart types 087 are selected (e.g., histogram, nonlinear probability curves, cumulative distribution S-curves, area charts, bar charts, etc.), named 088, notated 089, and saved 086. The simulation process is initiated 090, and the results are shown as a histogram bar chart 091. A chart overlay curve of the best-fitting distribution is also shown by default 092. The x-axis contains the numerical spread of the distribution, and the y-axis contains the frequency and probability density functions. The chart can be manipulated as desired (color, shading, rotation, skew, text, font, etc.) through a series of icon buttons 093. A selection of various distribution types (e.g., two tail, <left tail, ≧right tail, etc.) 094 is made with the certainty amounts entered 095 and the corresponding percentage confidence will be computed 097, or the desired percentage confidence is entered 097 and the corresponding certainty amount will be calculated 095. This process is initiated with the compute function 097. Reports are generated 098 and charts may be copied 099. Charts, statistics, chart data, and simulation data are generated and ready to be viewed 100.

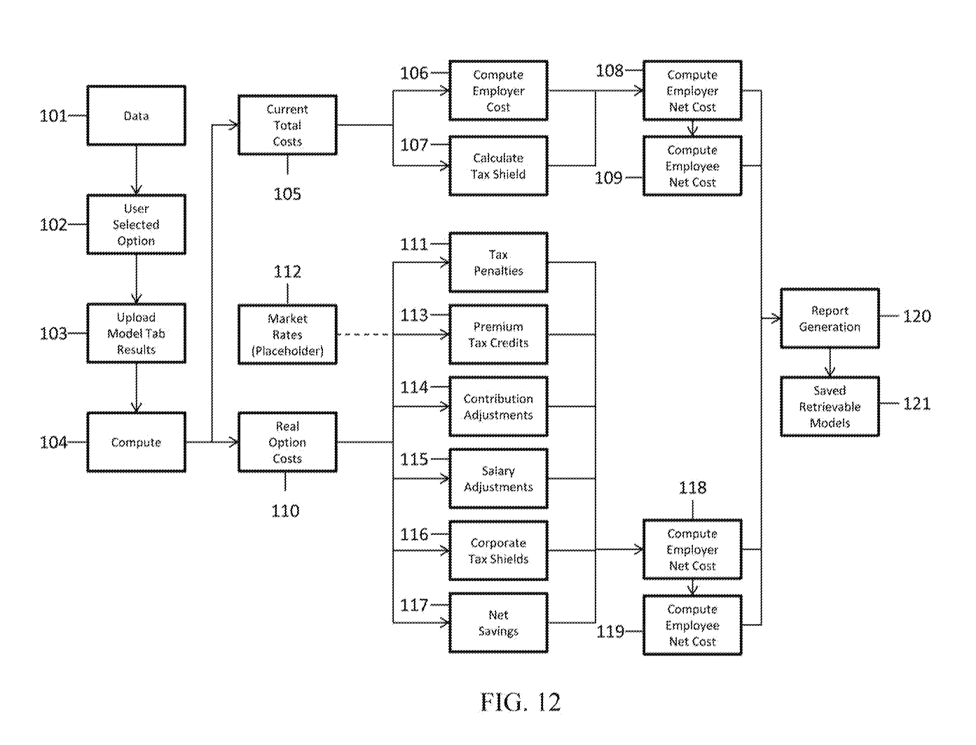

FIG. 12 illustrates the real options process, in accordance with an embodiment of the present invention, through a flowchart. The real options process begins with the mapping and validation of variables from the data table to their appropriate fields within the model 101. The algorithms and models selected option from the drop-down list are unique to each option selected 102 and apply only to employers with fifty or more full-time employees. The loading of the model tab results 103 and the computation process 104 are initiated by the user and accomplished in sequence. All calculations are system calculations performed within the utility and reported on the screen. The current total cost expenditure for the employer 105 is a calculation of the total employer and employee contributions for the total cost of health care. The employer cost 106 is calculated by reducing the current total cost expenditure by the aggregate amount of all employee contributions 107. The net employer cost 108 is a derivative of the employer cost 106 less the corporate tax savings 107 that result from the deductibility of employer-provided health insurance under Section §162 of the Internal Revenue Code. The present invention provides computations 109 for every employee under each option.

The tax penalty calculation 111 is an Affordable Care Act penalty for not providing health insurance to eligible employees. The algorithm determines the full- or part-time status of each individual based on variables in the data tables (i.e., reported employment status, full-time equivalency calculations that convert part-time employees into full-time equivalents etc., as noted in the algorithm above) and calculates the penalty amount the employer would be required to pay for each full-time equivalent as a nondeductible penalty tax if it elected not to provide employer-sponsored health insurance to its full-time employees.

Premium tax credits 113 were defined by the Affordable Care Act and are built into the algorithm for each appropriate option to determine the dollar amount each qualified employee could receive as a tax credit through the purchase of coverage in an Exchange. The Exchange is the only place where premium tax credits are available, and they are advance payments from the government paid through the Exchange mechanism to the insurance carrier or health maintenance organization on behalf of the qualified employee. The amount is based on income, number of dependents, and federal poverty level limits based on the state of residence. The process is to determine the percentage limits (as noted as % Income Table B in the diagram of household income allowable under the Affordable Care Act legislation) and calculate the maximum dollar threshold an employee could be compelled to contribute toward the cost of his or her health care and still meet the affordability requirement under the Affordable Care Act. The calculation process utilizes a placeholder proxy for coverage market rates 112 until each state's Exchange rating structure is linked into the utility. (This is presently under development with the Department of Health and Human Services and with a few of the state Exchanges. A Freedom of Information Act request has been initiated [Feb. 16, 2012] with the Centers for Medicare & Medicaid Services, Center for Consumer Information and Insurance Oversight requesting the release of the electronic master rate, carrier plan design data file that supports the individual and small business health-care offerings publicly available through the www.healthcare.gov website.) This premium tax credit calculation also was expanded to include the Medicaid-eligible population not eligible for premium tax credits, but who would be automatically enrolled in the Medicaid program through an eligibility determination process made by the Exchange. This represents a subsidy and is built in as a tax credit to get the most accurate representation of an employer's population in real economic terms. The premium tax credit calculation process is noted with each individual below 138% of the matched federal poverty level limit determined as auto-enrolled in Medicaid and allowed a credit in the amount of 100% of the placeholder proxy rate 112.

Contribution adjustments 114 are for those options that have a defined contribution limit or a change in plan pricing based on adopting a new design structure. This applies to the current options that are prebuilt and to expandable capability incorporated into the utility titled custom build option, which allows for the creation of unique grouping characteristics through SQL Data Filtering and Data Compute functions. An example of a contribution adjustment based on the adoption of a new design structure that results in a plan pricing change would be the consideration of a high-deductible health plan with the option to fund or not fund health savings accounts on behalf of the employees. An example of a contribution adjustment based on a defined contribution approach would be the consideration of a per person dollar limit provided by the employer to the employee for the purchase health care in the market.

Salary adjustments 115 are for those options that have a gross up feature. This refers to an increase in an employee's annual compensation by a designated amount as exemplified in a redirection of all or some portion of the savings resulting from the termination of the employer-sponsored health insurance plan to the employees in the form increased compensation.

Corporate tax shields 116 represent the combination of the effective corporate tax rate the employer pays (User Input), FICA (Federal Insurance Contributions Act used to fund Old Age Survivors Disability Insurance and Medicare), and FUTA (Federal Unemployment Tax Act). This calculation is made for each individual and is aggregated by dollar amount to reflect the impact by option.

Net savings 117 reflects the difference between the current employer net cost and each selected option's net employer cost. Total employer cost 118 reflects the cost of the selected option accounting for the full-time and part-time employee populations. Total employee cost 119 reflects the cost of the selected option accounting for the full-time and part-time employee populations. Results 120 are generated for tabular and graphical display with the capability to perform simulations. Models 121 may be saved and retrieved for each of the options analyses performed.

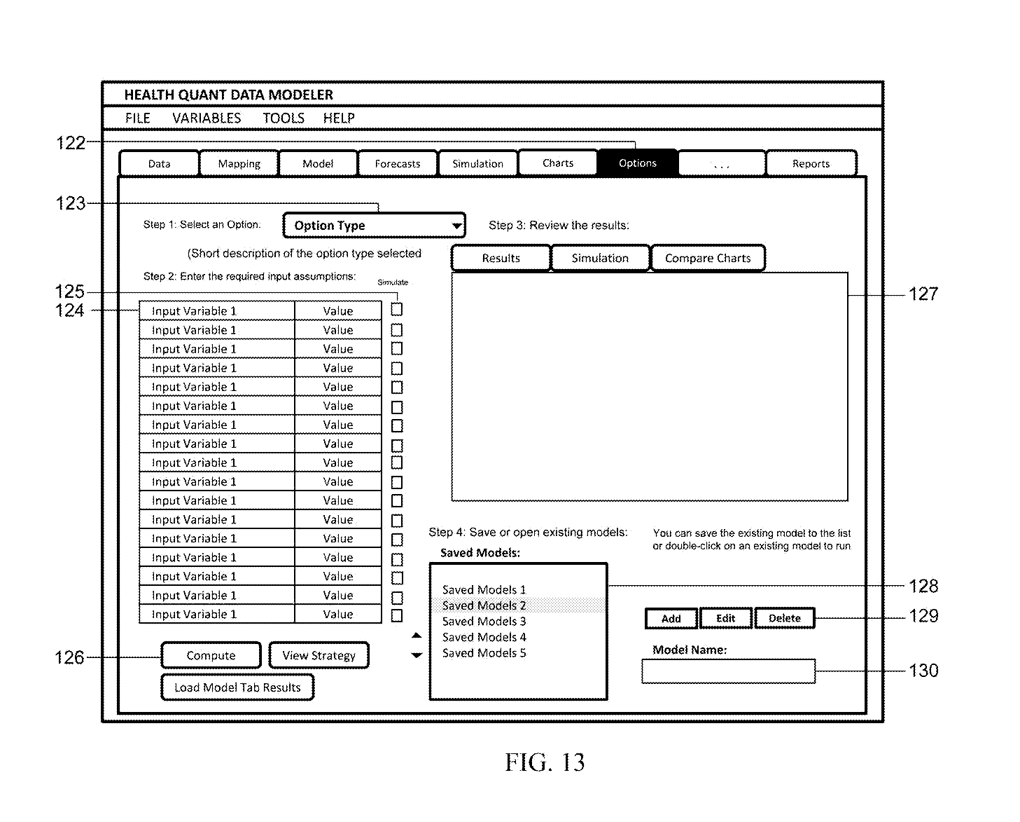

FIG. 13 further illustrates the real options process, in accordance with an embodiment of the present invention, through a user interface. The options tab is first selected 122 and the option type is chosen from among a drop-down list of strategic real options 123. This drop-down list contains prebuilt option types and is flexible to incorporate any type of combinations of strategies as previously described. The input variables from the data tables and data mapping processes are noted as input variables 124 and available for simulation 125. That is, instead of using the model computed results or single point estimates, a probability distribution or range of input assumptions can be set up and subjected to Monte Carlo risk simulation. The compute function 126 initiates the real options application and the results, simulations, and the resulting comparative charts can be viewed 127. Models can be named 130 and saved (edited and/or deleted) 129, and are retrievable 128 at a later date.

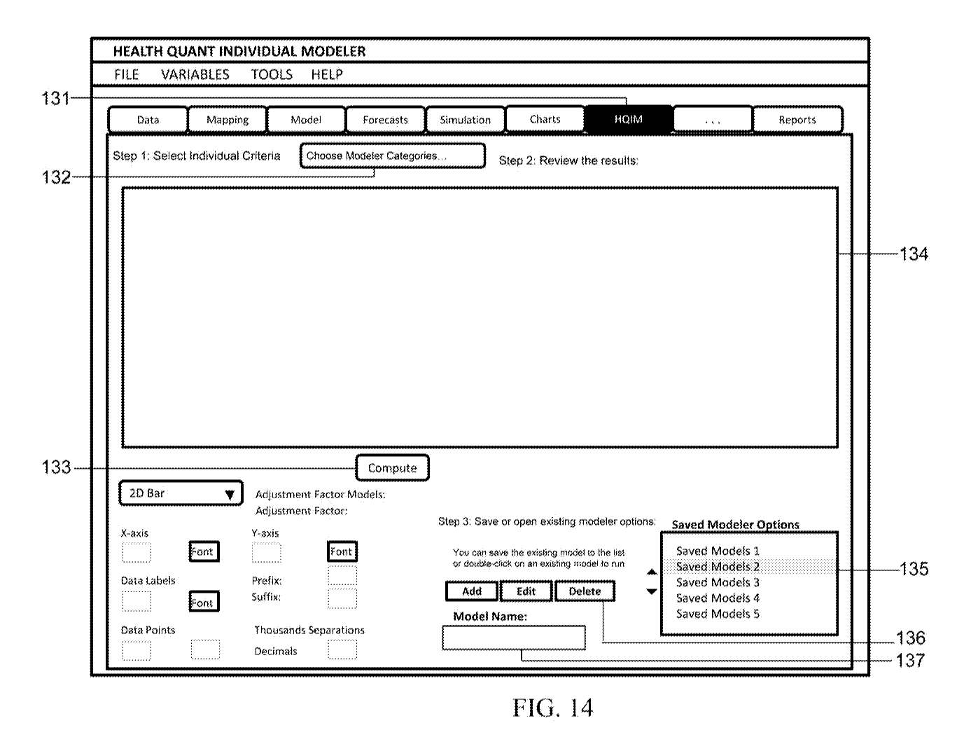

FIG. 14 illustrates the individual modeler process, in accordance with an embodiment of the present invention, through a user interface. The modeler application creates a unique individual custom-built option model by integrating the optimization, simulation, and real options analysis functionalities as described previously. The process is initiated by selecting the individual modeler function tab (HQIM) 131. The modeler categories 132 include a listing of options from among a drop-down list of options that populate the model with carrier names, plan names, plan levels, market rates, and design features. The user enters expected claims, tax rates, confidence levels desired, and constraints (e.g., budget constraints). The modeler will run a Monte Carlo risk simulation 133 and provide a graphical output of expected claims within the confidence intervals selected. An optimization is run that ranks each of the options ranging from noncompliance with penalty to the optimal match based on the confidence level chosen. The plans are ranked and graphed as a histogram or chart, with a supporting rates table 134. Models can be named 137 and saved (edited and/or deleted) 136, and are retrievable 135 at a future date.

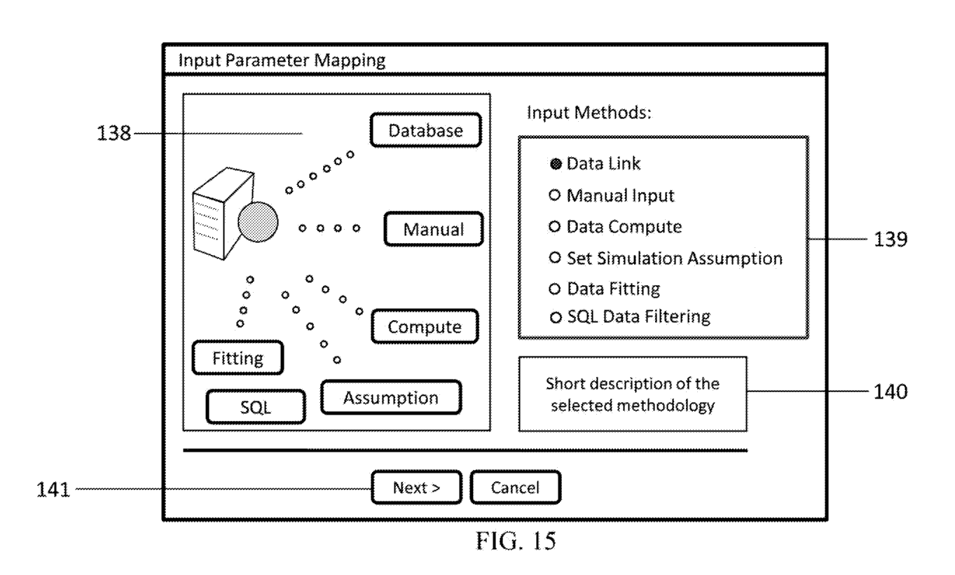

FIG. 15 illustrates the various input mapping parameters 138, in accordance with an embodiment of the present invention, where the radio buttons allow the desired method to be selected 139 and a short description of the selected methodology is shown 140. The various methods are data link (linking to existing data files, databases, and other proprietary data sources), manual input (data are typed in or pasted in directly), data compute (existing data variables are first modified and analyzed before being entered as input variables), set assumption (creating any of the twenty-four statistical distributions to run simulations on), model fitting (using existing raw data to find the best-fitting distribution assumption for simulation), and SQL data filtering (uses SQL expressions to build specific groups). The Next button proceeds to the selected method's next step 141.

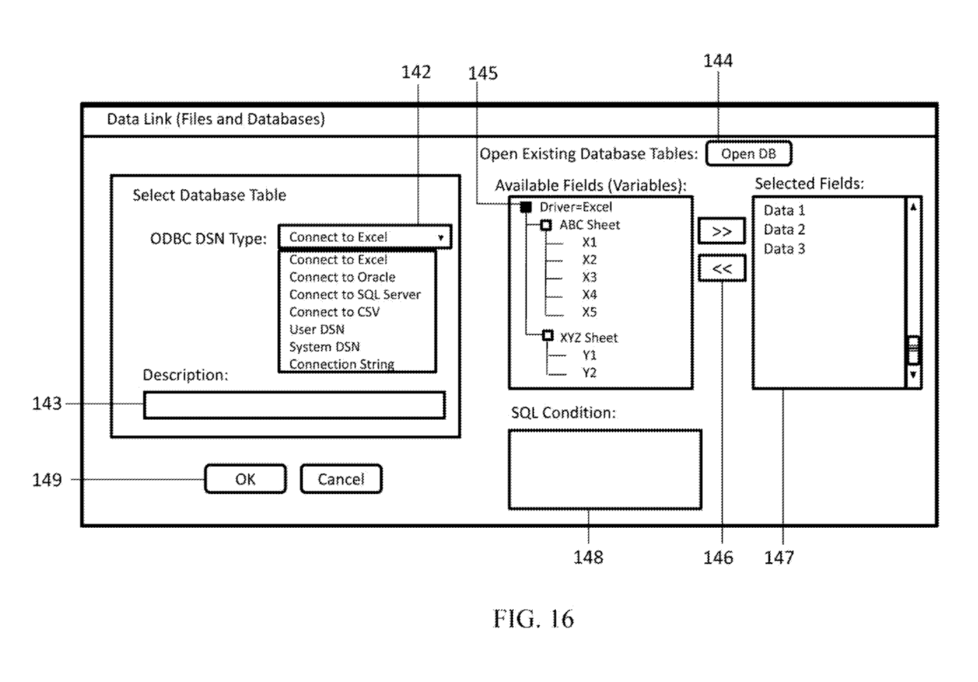

FIG. 16 illustrates the data link process, in accordance with an embodiment of the present invention, where an existing database, data file, or data table can be opened 142. The types of database will have to be ODBC-compliant database standards, text files, Excel files, CSV comma-delimited files, Oracle data files, or other custom databases as long as they are ODBC compliant. Depending on the database type selected, the relevant code-base descriptions or instructions are entered here (e.g., login, password, database file location) 143. The Open DB button will open the selected table types 144. Available field variables are noted in the respective root directory 145 and may be selected or deselected 146. After selecting the fields 147, the data can then be filtered using conditional SQL statements 148. Execution of the predefined instructions is then performed 149.

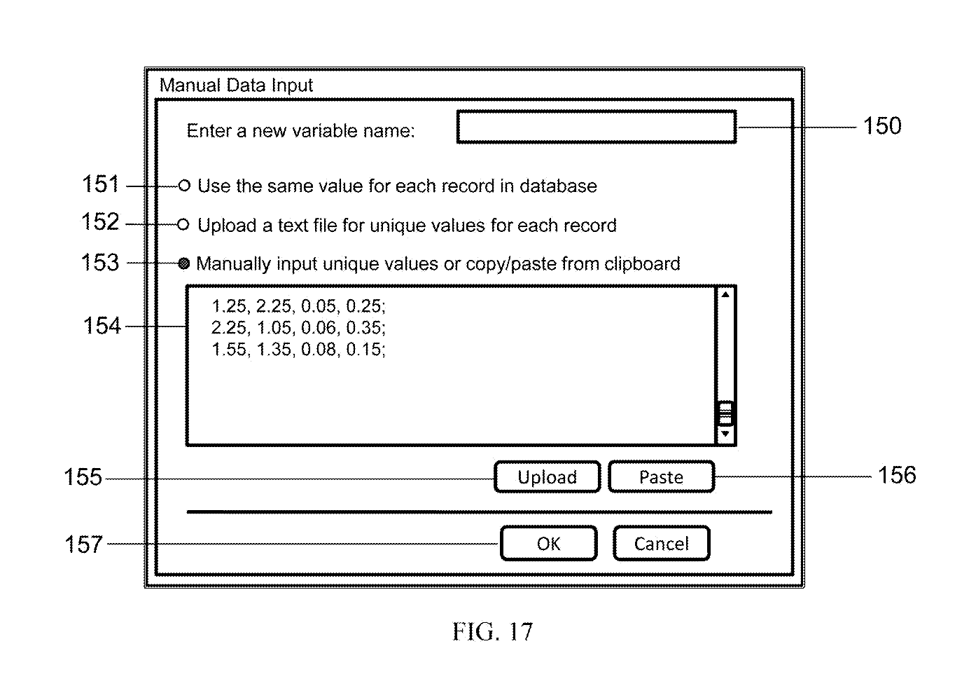

FIG. 17 illustrates the manual input process method, in accordance with an embodiment of the present invention, where a new data variable 150 can be entered in as a matrix, array, or sequence or uploaded from a flat data file, or a single value is replicated for every record in the variable. The tool allows you to manually input data for a required variable. A sample use would be when monthly claims data could be copied from a transactional claims payment system and pasted into the utility to allow for an expeditious transfer of data. A single number can be entered and applied through the entire database 151, or a text file for unique values for each record in the variable can be uploaded 152. The manual input process 153 provides a space 154 to enter the data (separated by a comma, tab, or space) where each row of the matrix is separated by a semicolon. The uploading 155 and pasting 156 is initiated or cancelled 157 and the new variable will be created.

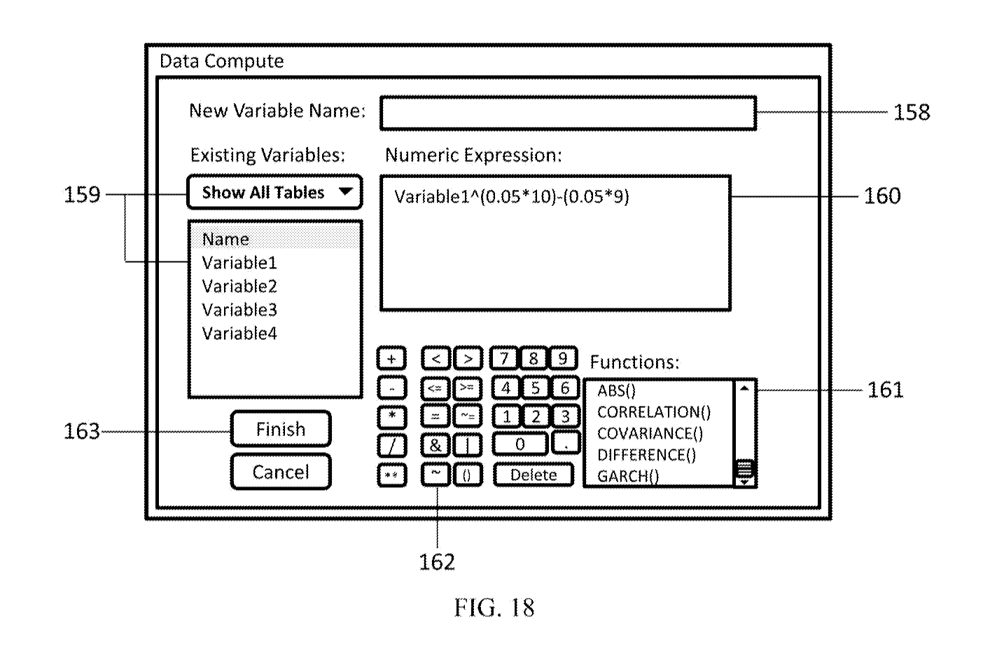

FIG. 18 illustrates the data compute method process, in accordance with an embodiment of the present invention, where existing variables can be used to compute and generate a new variable. This data computation method can parse mathematical functions as illustrated, including multiple mathematical, statistical, and financial functions and can be applied to numerical inputs typed in directly or using existing data variables. A sample use of this computation would be an increase in a per-member-per-month unit cost by a specified percentage to reflect an increase in the actuarial value of a benefit within a strategic business unit. It begins with providing a new variable name 158 for computation and drawing from existing variables within the available data tables 159. A numerical expression is built 160 to perform the required computation either by selecting from the existing functions 161 or by creating a function using the operators in the utility 162. The operation is then either executed or canceled 163.

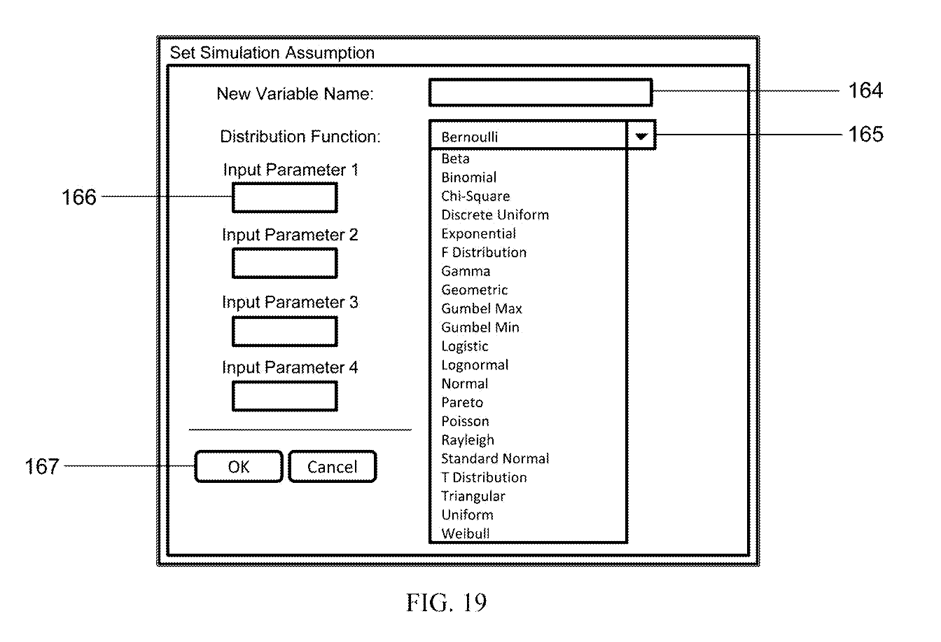

FIG. 19 illustrates an exemplary embodiment of the system's process of setting up various statistical distributions of an input variable for running simulations. Simulating variables continues to become an increasingly important component in analysis. A sample use of this functionality is in a typical claims forecast where the variables have different characteristics that need to simulated. For example, the enrollment of the firm might be quite stable (e.g., uniform distribution), claims experience might be quite normal (e.g., normal distribution), and large losses might be highly cyclical but limited by stop loss (e.g., custom distribution). The process begins with assigning a variable name 164, selecting the appropriate distribution 165, and entering the required parameters 166 based on the probability distribution selected. The operation is then either executed or canceled 167.

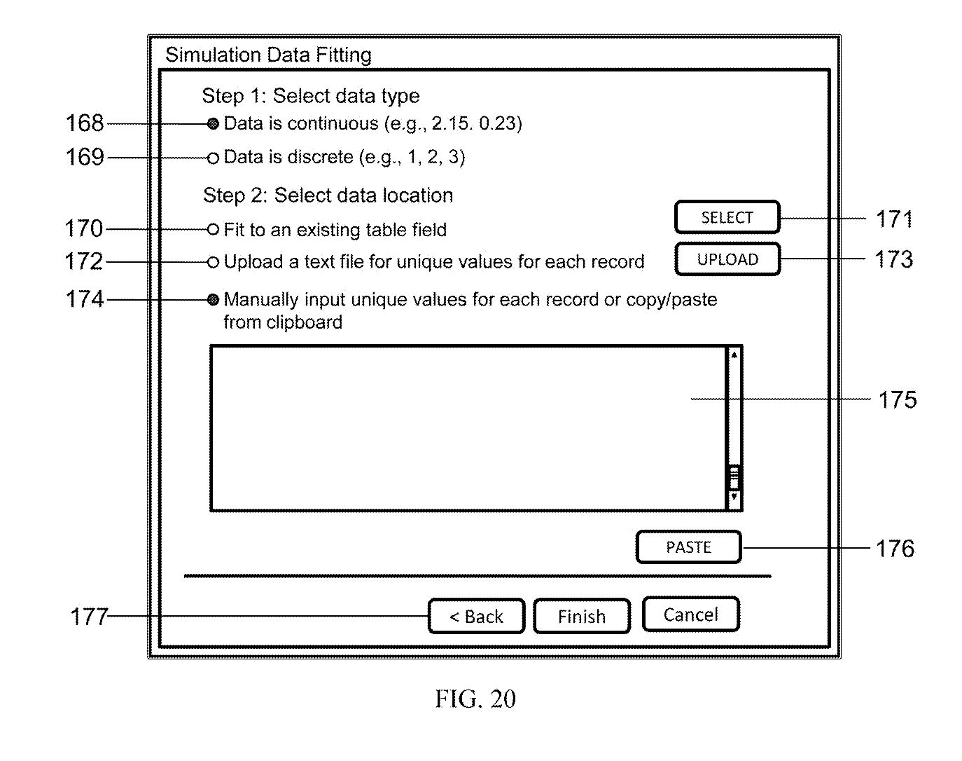

FIG. 20 illustrates an exemplary embodiment of the system's process of data model fitting of multiple data points to various statistical distributions of an input variable for running simulations. The tool is used to statistically test and fit existing data to the best probability distribution for running a Monte Carlo simulation. The first step is selecting whether the data is continuous data 168 (e.g., per-member-per-month unit cost to two decimals—$250.12) or discrete data 169 (e.g., 1,000 employees). The second step is to select the data location. One option is to fit the data to an existing table field 170 that allows for the opening of an existing database and SQL expression commands to allow the selection, verification, manipulation, and cleaning of the data before calculations take place 171. A second option is to upload 172 a text file for unique values for each record in the variable 173. A third option is a manual input process of unique values for each record or a copy/paste from a clipboard 174. Space is provided to write expressions 175 to capture data where the data must be separated by a comma, tab, or space and where each row of the matrix is separated by a semicolon, or paste the data 176. To go back, finish, or cancel is then initiated 177.

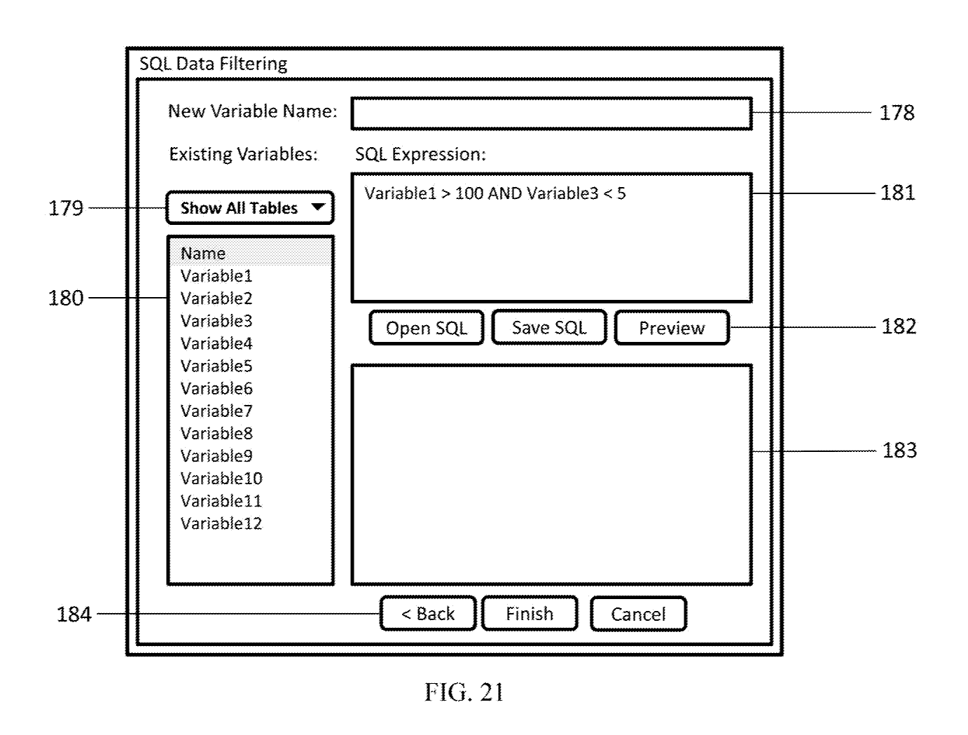

FIG. 21 illustrates an exemplary embodiment of the SQL data filtering method of generating new variables. An example of this critical capability is evident in custom-built real options applications where SQL expressions are written to map variables from among the various data tables. An instance of this example would be the creation of subgroups within a strategic business unit that were built to allow one option to apply to one subgroup, a different option to apply to a second subgroup, and a third unique option to apply to a third subgroup. It begins with the selection of the variable name 178, the selection of the data table 179, and the selection of the variables for the SQL Expression build 180. The SQL Expression is built 181, and a preview, opening, and saving of the SQL result is initiated 182. The result of the SQL Expression is displayed 183. To go back, finish, or cancel is then initiated 184.

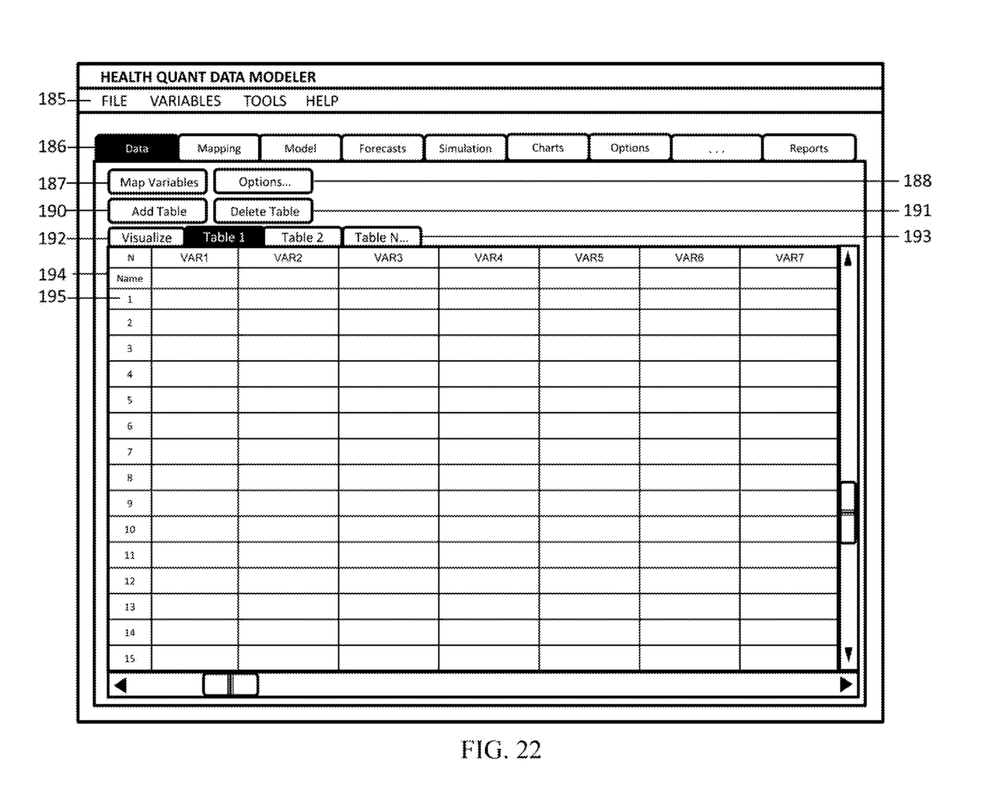

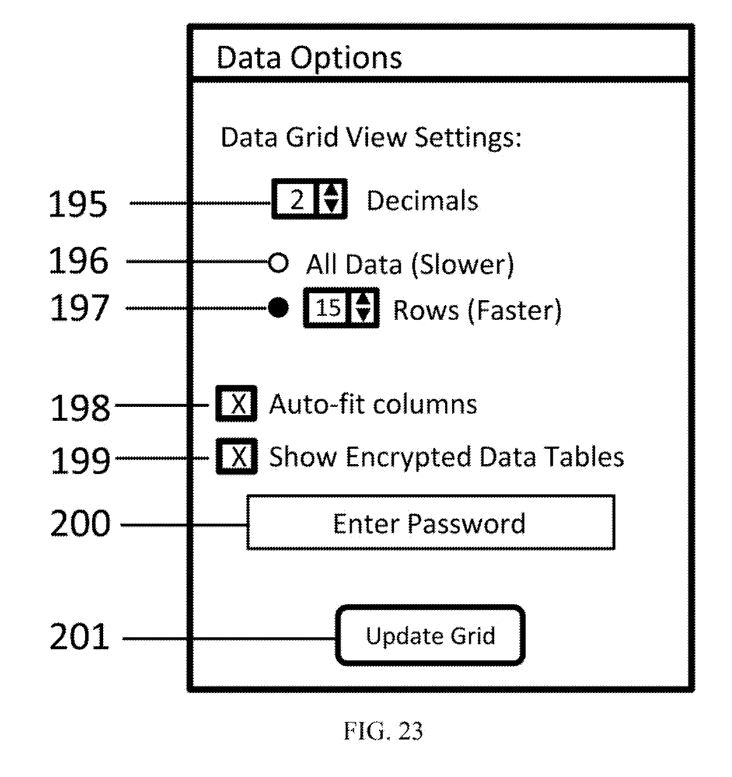

FIG. 22 and FIG. 23 illustrate an exemplary embodiment of the repository of data within the data tables. The data tables are the critical success factor in the model. Accessing the file 185 and the data table 186 creates the mapping variable function 187 and the mapping options 188 such as data link, manual input, data compute, simulation assumption, data fitting, and SQL data filtering. Tables may be added 190 or deleted 191. The data may be viewed graphically 192. There is no limit to the number of data tables that can be created 193. The variables are given names 194 with a specific record address 195. The data grid view settings for the data tables consist of setting the number of decimals 195, the number of data rows 197, and whether all of the data 196 or some of the data 197 is to be used. Columns can be auto-fitted to the data 198. Encrypted tables may be shown 199. Password requirements may be required and entered 200. To execute the settings, the grid is updated 201.

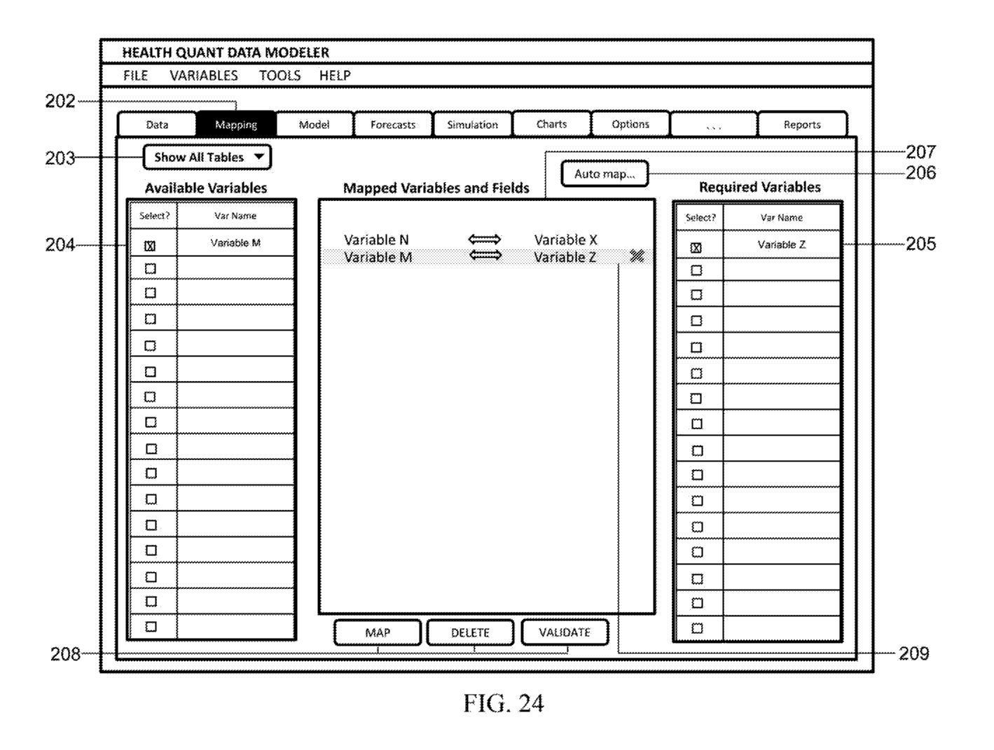

FIG. 24 illustrates an exemplary embodiment of the data mapping process through a user interface. A simple mapping example would be the routing of an employee with four covered dependents covered as a family in a three-tier rating structure (available variable) to an employee plus spouse and two children and in a five-tier rating structure (required variable). This process is initiated by selecting the mapping function 202, the data tables 203, and the available variables 204 from within the respective data tables. The mapping process is the link of the available variables from the data tables to the required variables within the models 205 used for proprietary data computation. An automatic mapping function that matches available variables 204 to required variables 206 has been built where the success of the routing is noted 209. To map 207, delete, and validate the mapping functionality, the execution function is noted 208.

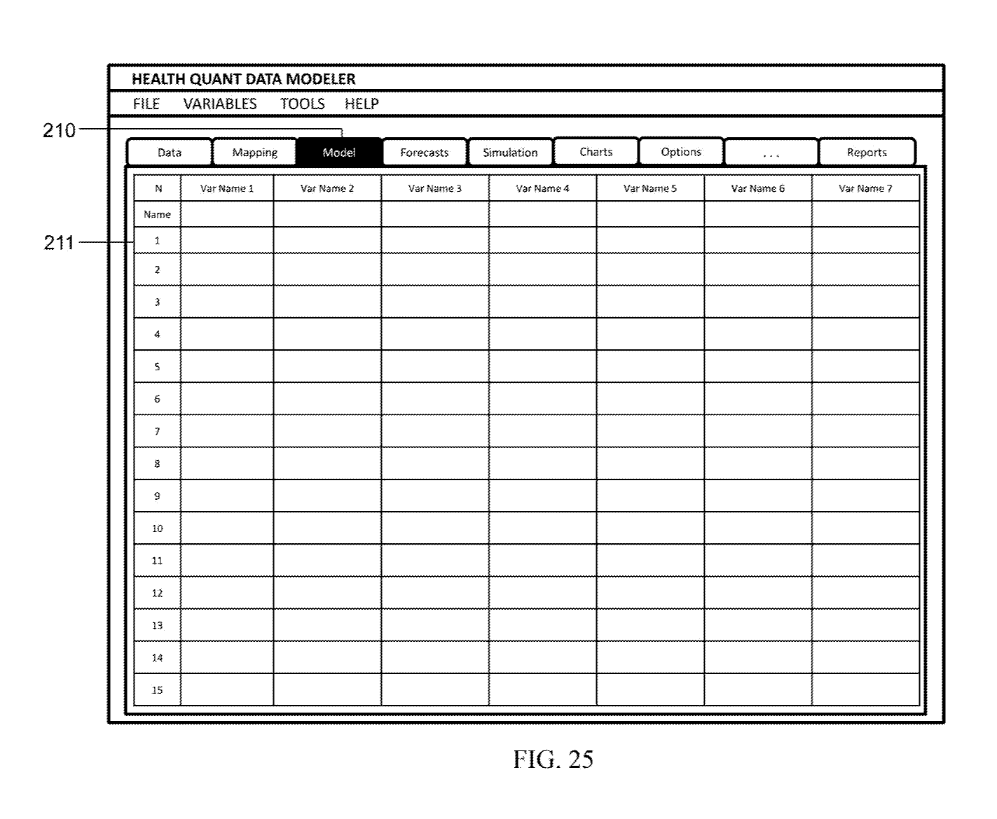

FIG. 25 illustrates an exemplary embodiment of the beginning of the Model through the user interface. The Model begins by selecting the Model tab 210 and reviewing the data table 211 created from the mapping process that lists the variable field names and is the repository of the data.

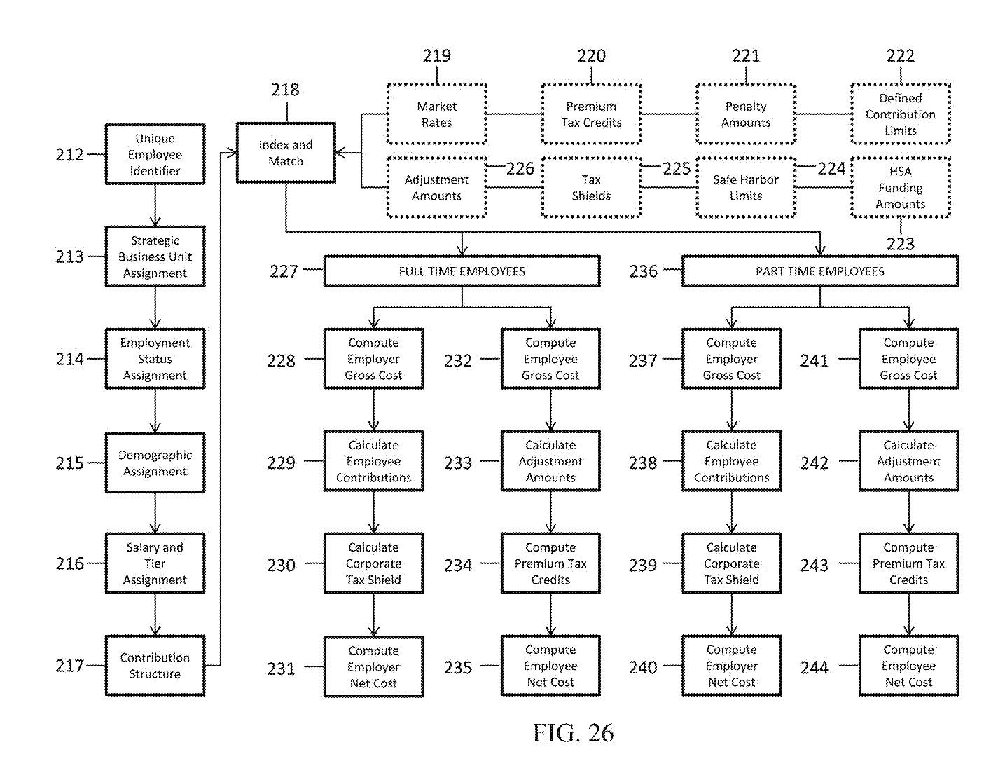

FIG. 26 illustrates an exemplary embodiment of the continuation of the Model through a flowchart. The Model starts by indexing each record based on a unique identification dimension such as unique employee identifier or member identification number 212. It then matches this dimension with additional dimensions such as strategic business unit 213 (e.g., a line of business, division, subsidiary, etc.); employment status 214 (e.g., full time, part time, union, non-union, hourly, salaried, exempt, nonexempt); demographic information 215 (e.g., age, gender, number of dependents, state of residence, etc.); payroll 216; and election information 217 (e.g., salary and health plan election, and structure of employer and employee contributions). The Model then matches each multidimensional record with facts sourced from internal data tables such as market rates 219, premium tax credits 220, penalty amounts 221, defined contribution limits 222, health savings account funding amounts 223, safe harbor employee contribution limits 224, calculated lost tax shields 225, compensation amounts under certain options 226, and the like for option calculation purposes. Each option calculation is performed for full-time employees 227 and consists of two sets of computations. The first is done to compute employer net cost 231 and the second is done to compute the employee net cost 235. Employer net cost 231 is computed by subtracting employee contributions 229 and lost corporate tax shields 230 from the employer gross cost 228. Employee net cost 235 is computed by subtracting gross up amounts 233 and premium tax credits 234 from the employee gross cost 232. Each option calculation is performed for part-time employees 236 and consists of two sets of computations. The first is done to compute employer net cost 240 and the second is done to compute the employee net cost 244. Employer net cost 240 is computed by subtracting employee contributions 238 and lost corporate tax shields 239 from the employer gross cost 237. Employee net cost 244 is computed by subtracting gross up amounts 242 and premium tax credits 243 from the employee gross cost 241.

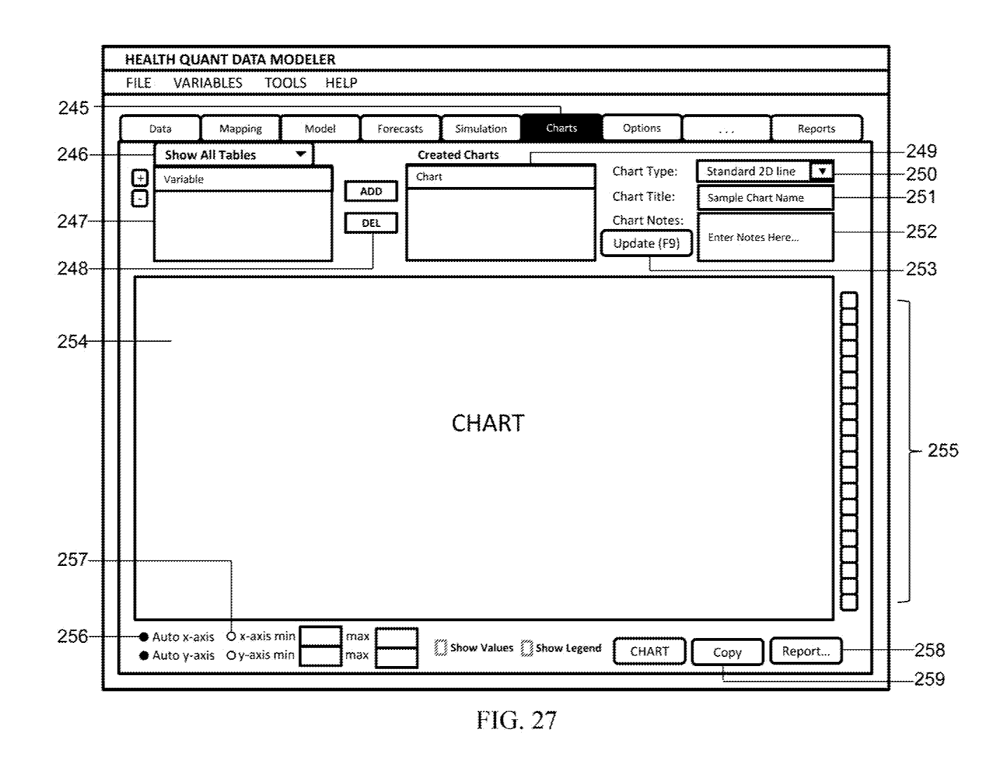

FIG. 27 illustrates an exemplary embodiment of the charting capabilities. It is initiated by selecting the chart tab 245 and tables 246. The variables are chosen 247 and charts are added and/or deleted 248. Saved charts may be retrieved 249. Different chart types may be selected (control charts, histograms, line charts, etc.) 250. Chart name 251 and chart notes 252 are provided. An example would be a control chart for large dollar claim amounts that are compared to one, two, and three sigma levels calculated above and below the computed control limit. The chart is updated 253 and the graphical results are displayed 254 with additional presentation graphics available 255. Automatic axis scaling and formatting of the x-axis and y-axis is noted 256. A manual scaling with minimum and maximum amounts is available 257. A report may be selected and run 258. A chart may be copied 259 and pasted to another application.

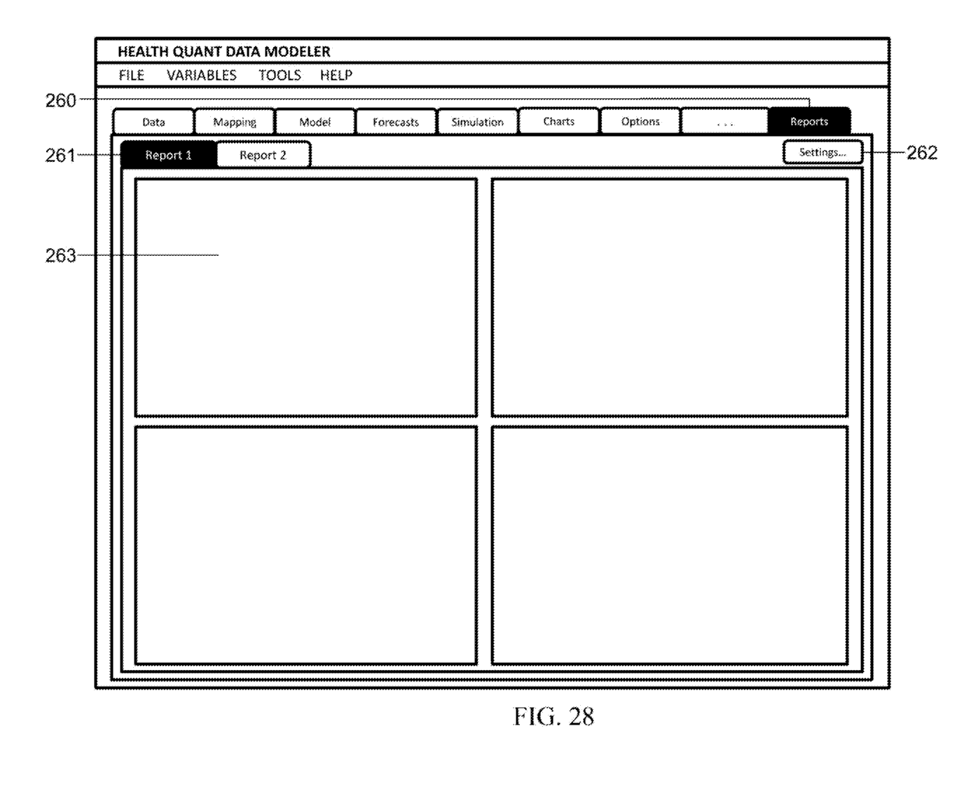

FIG. 28 illustrates an exemplary embodiment of the reporting capabilities of the present invention. A sample application of this capability is represented by a dashboard application that could display unique graphs in each of the four quadrants that provide an executive summary of key performance metrics. An example of this application would be the reporting compliance percentages for evidence-based medicine (quadrant one), risk score distributions for an employer's population (quadrant two), disease state incidence rates per 1,000 members (quadrant three), and forecasted claims amounts (quadrant four). Each of these quadrants has the flexibility to be exchanged for a different key performance metric with its graph and can also be saved as a retrievable dashboard. The process begins with selecting the Reports tab 260 along with each of the quadrant's key performance metrics to be reported in the dashboard 261 and the settings 262 in each quadrant can be changed (e.g., chart types, results of which model to show, and so forth). The results are reported in their respective quadrants 263.

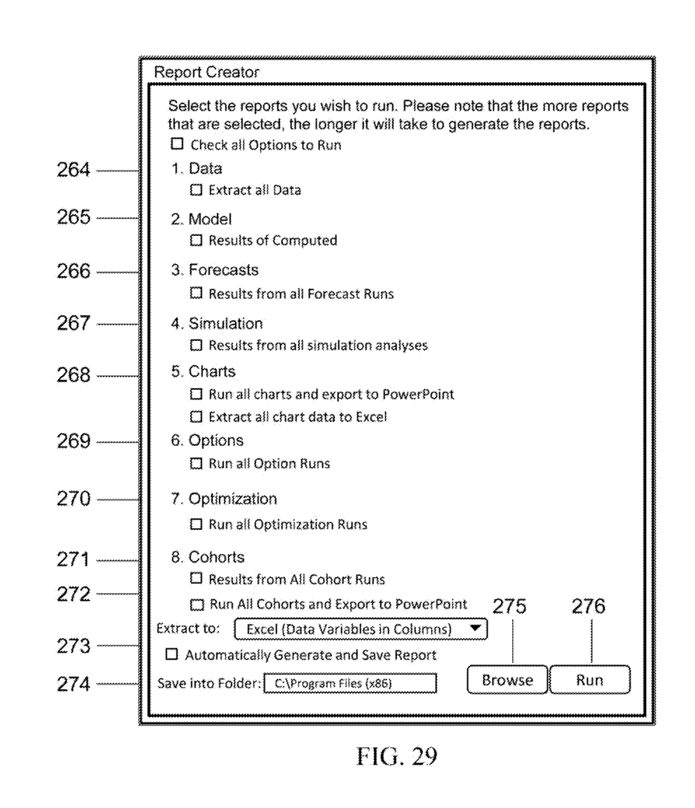

FIG. 29 illustrates an exemplary embodiment of the report creator functionality of the present invention. This application allows the user to export results from each chosen function into Excel. Each of the following elements may be exported: all data for each data table 264; results of the computation from the model 265; each and all forecast runs 266; Monte Carlo simulation results 267; charts exported to PowerPoint with the data exported into an Excel spreadsheet 268; all or selected options 269; optimization models 270; and cohort results 271 with charts exported to PowerPoint with the data exported into an Excel spreadsheet 272. An automatic report generator and saving function 273 is set up to allow for the saving of the files into a default folder 274. The browse functionality permits the saving of the reports to a separate file folder location 275. The run command 276 executes the selected report extracts.

Mathematical Probability Distributions

This section demonstrates the mathematical models and computations used in creating the Monte Carlo risk-based simulations as described throughout the current invention. In order to get started with simulation, one first needs to understand the concept of probability distributions. To begin to understand probability, consider this example: You want to look at the distribution of nonexempt wages within one department of a large company. First, you gather raw data—in this case, the wages of each nonexempt employee in the department. Second, you organize the data into a meaningful format and plot the data as a frequency distribution on a chart. To create a frequency distribution, you divide the wages into group intervals and list these intervals on the chart's horizontal axis. Then you list the number or frequency of employees in each interval on the chart's vertical axis. Now you can easily see the distribution of nonexempt wages within the department. You can chart this data as a probability distribution. A probability distribution shows the number of employees in each interval as a fraction of the total number of employees. To create a probability distribution, you divide the number of employees in each interval by the total number of employees and list the results on the chart's vertical axis.

Probability distributions are either discrete or continuous. Discrete probability distributions describe distinct values, usually integers, with no intermediate values and are shown as a series of vertical bars. A discrete distribution, for example, might describe the number of heads in four flips of a coin as 0, 1, 2, 3, or 4. Continuous probability distributions are actually mathematical abstractions because they assume the existence of every possible intermediate value between two numbers; that is, a continuous distribution assumes there is an infinite number of values between any two points in the distribution. However, in many situations, you can effectively use a continuous distribution to approximate a discrete distribution even though the continuous model does not necessarily describe the situation exactly.

Probability Density Functions, Cumulative Distribution Functions, and Probability Mass Functions

In mathematics and Monte Carlo simulation, a probability density function (PDF) represents a continuous probability distribution in terms of integrals. If a probability distribution has a density of ƒ(x), then intuitively the infinitesimal interval of [x, x+dx] has a probability of ƒ(x)dx. The PDF therefore can be seen as a smoothed version of a probability histogram; that is, by providing an empirically large sample of a continuous random variable repeatedly, the histogram using very narrow ranges will resemble the random variable's PDF. The probability of the interval between [a, b] is given by

$${\int }_{a}^{b} f ( x ) d x ,$$

which means that the total integral of the function ƒ must be 1.0. It is a common mistake to think of ƒ(a) as the probability of a. This is incorrect. In fact, ƒ(a) can sometimes be larger than 1—consider a uniform distribution between 0.0 and 0.5. The random variable x within this distribution will have ƒ(x) greater than 1. The probability in reality is the function ƒ(x)dx discussed previously, where dx is an infinitesimal amount.

The cumulative distribution function (CDF) is denoted as F(x)=P(X≦x) indicating the probability of X taking on a less than or equal value to x. Every CDF is monotonically increasing, is continuous from the right, and at the limits, have the following properties:

$${lim}_{x \to - ∞} F ( x ) = 0 and {lim}_{x \to + ∞} F ( x ) = 1.$$

Further, the CDF is related to the PDF by

$$F ( b ) - F ( a ) = P ( a \leq X \leq b ) = {\int }_{a}^{b} f ( x ) d x ,$$

where the PDF function ƒ is the derivative of the CDF function F.

In probability theory, a probability mass function or PMF gives the probability that a discrete random variable is exactly equal to some value. The PMF differs from the PDF in that the values of the latter, defined only for continuous random variables, are not probabilities; rather, its integral over a set of possible values of the random variable is a probability. A random variable is discrete if its probability distribution is discrete and can be characterized by a PMF. Therefore, X is a discrete random variable if

$${\sum }_{u} P ( X = u ) = 1$$

as u runs through all possible values of the random variable X.

Discrete Distributions

Following is a detailed listing of the different types of probability distributions that can be used in Monte Carlo simulation.

Bernoulli or Yes/No Distribution

The Bernoulli distribution is a discrete distribution with two outcomes (e.g., head or tails, success or failure, 0 or 1). The Bernoulli distribution is the binomial distribution with one trial and can be used to simulate Yes/No or Success/Failure conditions. This distribution is the fundamental building block of other more complex distributions. For instance:
Binomial distribution: Bernoulli distribution with higher number of n total trials and computes the probability of x successes within this total number of trials.
Geometric distribution: Bernoulli distribution with higher number of trials and computes the number of failures required before the first success occurs.
Negative binomial distribution: Bernoulli distribution with higher number of trials and computes the number of failures before the xth success occurs.

The mathematical constructs for the Bernoulli distribution are as follows:

$$P ( x ) = \{ \begin{matrix}1 - p & for x = 0 \\ p & for x = 1\end{matrix} \\ or \\ P ( x ) = {{p}^{x} ( 1 - p )}^{1 - x} \\ mean = p \\ standard deviation = \sqrt{p ( 1 - p )} \\ skewness = \frac{1 - 2 p}{\sqrt{p ( 1 - p )}} \\ excess kurtosis = \frac{6 {p}^{2} - 6 p + 1}{p ( 1 - p )}$$

The probability of success (p) is the only distributional parameter. Also, it is important to note that there is only one trial in the Bernoulli distribution, and the resulting simulated value is either 0 or 1. The input requirements are such that Probability of Success >0 and <1 (that is, 0.0001≦p≦0.9999).

Binomial Distribution

The binomial distribution describes the number of times a particular event occurs in a fixed number of trials, such as the number of heads in 10 flips of a coin or the number of defective items out of 50 items chosen.

The three conditions underlying the binomial distribution are:
For each trial, only two outcomes are possible that are mutually exclusive.
The trials are independent—what happens in the first trial does not affect the next trial.
The probability of an event occurring remains the same from trial to trial.

The mathematical constructs for the binomial distribution are as follows:

$$P ( x ) = \frac{n !}{x ! ( n - x ) !} {{p}^{x} ( 1 - p )}^{( n - x )}$$

$$for n > 0 ; x = 0 , 1 , 2 , … n ; and 0 < p < 1$$

$$mean = np$$

$$standard deviation = \sqrt{np ( 1 - p )}$$

$$skewness = \frac{1 - 2 p}{\sqrt{np ( 1 - p )}}$$

$$excess kurtosis = \frac{6 {p}^{2} - 6 p + 1}{np ( 1 - p )}$$

The probability of success (p) and the integer number of total trials (n) are the distributional parameters. The number of successful trials is denoted x. It is important to note that probability of success (p) of 0 or 1 are trivial conditions and do not require any simulations, and hence, are not allowed in the software. The input requirements are such that Probability of Success >0 and <1 (that is, 0.0001≦p≦0.9999), the Number of Trials ≧1 or positive integers and ≦1000 (for larger trials, use the normal distribution with the relevant computed binomial mean and standard deviation as the normal distribution's parameters).

Discrete Uniform

The discrete uniform distribution is also known as the equally likely outcomes distribution, where the distribution has a set of N elements, and each element has the same probability. This distribution is related to the uniform distribution but its elements are discrete and not continuous.

The mathematical constructs for the discrete uniform distribution are as follows:

$$P ( x ) = \frac{1}{N}$$

$$mean = \frac{N + 1}{2} ranked value$$

$$standard deviation = \sqrt{\frac{( N - 1 ) ( N + 1 )}{12}} ranked value$$

$$skewness = 0 ( that is , the distribution is perfectly symmetrical )$$

$$excess kurtosis = \frac{- 6 ( {N}^{2} + 1 )}{5 ( N - 1 ) ( N + 1 )} ranked value$$

The input requirements are such that Minimum<Maximum and both must be integers (negative integers and zero are allowed).

Geometric Distribution

The geometric distribution describes the number of trials until the first successful occurrence, such as the number of times you need to spin a roulette wheel before you win.

The three conditions underlying the geometric distribution are:
The number of trials is not fixed.
The trials continue until the first success.
The probability of success is the same from trial to trial.

The mathematical constructs for the geometric distribution are as follows:

$$P ( x ) = {p ( 1 - p )}^{x - 1} for 0 < p < 1 and x = 1 , 2 , … , n$$

$$mean = \frac{1}{p} - 1$$

$$standard deviation = \sqrt{\frac{1 - p}{{p}^{2}}}$$

$$skewness = \frac{2 - p}{\sqrt{1 - p}}$$

$$excess kurtosis = \frac{{p}^{2} - 6 p + 6}{1 - p}$$

The probability of success (p) is the only distributional parameter. The number of successful trials simulated is denoted x, which can only take on positive integers. The input requirements are such that Probability of success >0 and <1 (that is, 0.0001≦p≦0.9999). It is important to note that probability of success (p) of 0 or 1 are trivial conditions and do not require any simulations, and hence, are not allowed in the software.

Hypergeometric Distribution

The hypergeometric distribution is similar to the binomial distribution in that both describe the number of times a particular event occurs in a fixed number of trials. The difference is that binomial distribution trials are independent, whereas hypergeometric distribution trials change the probability for each subsequent trial and are called trials without replacement. For example, suppose a box of manufactured parts is known to contain some defective parts. You choose a part from the box, find it is defective, and remove the part from the box. If you choose another part from the box, the probability that it is defective is somewhat lower than for the first part because you have removed a defective part. If you had replaced the defective part, the probabilities would have remained the same, and the process would have satisfied the conditions for a binomial distribution.

The three conditions underlying the hypergeometric distribution are:
The total number of items or elements (the population size) is a fixed number, a finite population. The population size must be less than or equal to 1,750.
The sample size (the number of trials) represents a portion of the population.
The known initial probability of success in the population changes after each trial.

The mathematical constructs for the hypergeometric distribution are as follows:

$$P ( x ) = \frac{\frac{( {N}_{x} ) !}{x ! ( {N}_{x} - x ) !} \frac{( N - {N}_{x} ) !}{( n - x ) ! ( N - {N}_{x} - n + x ) !}}{\frac{N !}{n ! ( N - n ) !}}$$

$$for x = Max ( n - ( N - {N}_{x} ) , 0 ) , … , Min ( n , {N}_{x} )$$

$$mean = \frac{{N}_{x} n}{N}$$

$$standard deviation = \sqrt{\frac{( N - {N}_{x} ) {N}_{x} n ( N - n )}{{N}^{2} ( N - 1 )}}$$

$$skewness = \frac{( N - {2}_{x} ) ( N - 2 n )}{N - 2} \sqrt{\frac{N - 1}{( N - {N}_{x} ) {N}_{x} n ( N - n )}}$$

$$excess kurtosis = \frac{V ( N , {N}_{x} , n )}{( N - {N}_{x} ) {N}_{x} n ( - 3 + N ) ( - 2 + N ) ( - N + n )}$$

$$where$$

$$V ( N , {N}_{x} , n ) = {( N - {N}_{x} )}^{3} - {( N - {N}_{x} )}^{5} + 3 {( N - {N}_{x} )}^{2} - 6 {( N - {N}_{x} )}^{3} {N}_{x} + {( N - {N}_{x} )}^{4} {N}_{x} + 3 ( N - {N}_{x} ) {N}_{x}^{2} - 12 {( N - {N}_{x} )}^{2} {N}_{x}^{2} + 8 {( N - {N}_{x} )}^{3} {N}_{x}^{2} + {N}_{x}^{3} - 6 ( N - {N}_{x} ) {N}_{x}^{3} + 8 {( N - {N}_{x} )}^{2} {N}_{x}^{3} + ( N - {N}_{x} ) {N}_{x}^{4} - {N}_{x}^{5} - 6 {( N - {N}_{x} )}^{3} {N}_{x} + 6 {( N - {N}_{x} )}^{4} {N}_{x} + 18 {( N - {N}_{x} )}^{2} {N}_{x} n - 6 {( N - {N}_{x} )}^{3} {N}_{x} n + 18 ( N - {N}_{x} ) {N}_{x}^{2} n - 24 {( N - {N}_{x} )}^{2} {N}_{x}^{2} n - 6 {( N - {N}_{x} )}^{3} n - 6 ( N - {N}_{x} ) {N}_{x}^{3} n + 6 {N}_{x}^{4} n + 6 {( N - {N}_{x} )}^{2} {n}^{2} - 6 {( N - {N}_{x} )}^{3} {n}^{2} - 24 ( N - {N}_{x} ) {N}_{x} {n}^{2} + 12 {( N - {N}_{x} )}^{2} {N}_{x} {n}^{2} + 6 {N}_{x}^{2} {n}^{2} + 12 ( N - {N}_{x} ) {N}_{x}^{2} {n}^{2} - 6 {N}_{x}^{3} {n}^{2}$$

The number of items in the population (N), trials sampled (n), and number of items in the population that have the successful trait (Nₓ) are the distributional parameters. The number of successful trials is denoted x. The input requirements are such that Population ≧2 and integer, Trials >0 and integer, Successes >0 and integer, Population>Successes, Trials<Population, and Population <1750.

Negative Binomial Distribution

The negative binomial distribution is useful for modeling the distribution of the number of trials until the rth successful occurrence, such as the number of sales calls you need to make to close a total of 10 orders. It is essentially a superdistribution of the geometric distribution. This distribution shows the probabilities of each number of trials in excess of r to produce the required success r.

Conditions

The three conditions underlying the negative binomial distribution are:
The number of trials is not fixed.
The trials continue until the rth success.
The probability of success is the same from trial to trial.

The mathematical constructs for the negative binomial distribution are as follows:

$$P ( x ) = \frac{( x + r - 1 ) !}{( r - 1 ) ! x !} {{p}^{r} ( 1 - p )}^{x}$$

$$for x = r , r + 1 , … ; and 0 < p < 1$$

$$mean = \frac{r ( 1 - p )}{p}$$

$$standard deviation = \sqrt{\frac{r ( 1 - p )}{{p}^{2}}}$$

$$skewness = \frac{2 - p}{\sqrt{r ( 1 - p )}}$$

$$excess kurtosis = \frac{{p}^{2} - 6 p + 6}{r ( 1 - p )}$$

Probability of success (p) and required successes (r) are the distributional parameters. Where the input requirements are such that Successes required must be positive integers >0 and <8000, Probability of success >0 and <1 (that is, 0.0001≦p≦0.9999). It is important to note that probability of success (p) of 0 or 1 are trivial conditions and do not require any simulations, and hence, are not allowed in the software.

Poisson Distribution

The Poisson distribution describes the number of times an event occurs in a given interval, such as the number of telephone calls per minute or the number of errors per page in a document.

Conditions

The three conditions underlying the Poisson distribution are:
The number of possible occurrences in any interval is unlimited.
The occurrences are independent. The number of occurrences in one interval does not affect the number of occurrences in other intervals.
The average number of occurrences must remain the same from interval to interval.

The mathematical constructs for the Poisson are as follows:

$$P ( x ) = \frac{{e}^{- λ} {λ}^{x}}{x !} for x and λ > 0$$

$$mean = λ$$

$$standard deviation = \sqrt{λ} \\ skewness = \frac{1}{\sqrt{λ}} \\ excess kurtosis = \frac{1}{λ}$$

Rate (λ) is the only distributional parameter and the input requirements are such that Rate >0 and ≦1000 (that is, 0.0001≦rate≦1000).

Continuous Distributions

Beta Distribution

The beta distribution is very flexible and is commonly used to represent variability over a fixed range. One of the more important applications of the beta distribution is its use as a conjugate distribution for the parameter of a Bernoulli distribution. In this application, the beta distribution is used to represent the uncertainty in the probability of occurrence of an event. It is also used to describe empirical data and predict the random behavior of percentages and fractions, as the range of outcomes is typically between 0 and 1. The value of the beta distribution lies in the wide variety of shapes it can assume when you vary the two parameters, alpha and beta. If the parameters are equal, the distribution is symmetrical. If either parameter is 1 and the other parameter is greater than 1, the distribution is J-shaped. If alpha is less than beta, the distribution is said to be positively skewed (most of the values are near the minimum value). If alpha is greater than beta, the distribution is negatively skewed (most of the values are near the maximum value). The mathematical constructs for the beta distribution are as follows:

$$f ( x ) = \frac{{( x )}^{( α - 1 )} {( 1 - x )}^{( β - 1 )}}{[ \frac{Γ ( α ) Γ ( β )}{Γ ( α + β )} ]} for α > 0 ; β > 0 ; x > 0$$

$$mean = \frac{α}{α + β}$$

$$standard deviation = \sqrt{\frac{α β}{{( α + β )}^{2} ( 1 + α + β )}}$$

$$skewness = \frac{2 ( β - α ) \sqrt{1 + α + β}}{( 2 + α + β ) \sqrt{α β}}$$

$$excess kurtosis = \frac{3 ( α + β + 1 ) [ \begin{matrix}α β ( α + β - 6 ) + \\ 2 {( α + β )}^{2}\end{matrix} ]}{α β ( α + β + 2 ) ( α + β + 3 )} - 3$$

Alpha (α) and beta (β) are the two distributional shape parameters, and Γ is the gamma function. The two conditions underlying the beta distribution are:
The uncertain variable is a random value between 0 and a positive value.
The shape of the distribution can be specified using two positive values.

Input requirements: Alpha and beta >0 and can be any positive value

Cauchy Distribution or Lorentzian Distribution or Breit-Wigner Distribution

The Cauchy distribution, also called the Lorentzian distribution or Breit-Wigner distribution, is a continuous distribution describing resonance behavior. It also describes the distribution of horizontal distances at which a line segment tilted at a random angle cuts the x-axis.

The mathematical constructs for the cauchy or Lorentzian distribution are as follows:

$$f ( x ) = \frac{1}{π} \frac{γ / 2}{{( x - m )}^{2} + {γ}^{2} / 4}$$

The cauchy distribution is a special case where it does not have any theoretical moments (mean, standard deviation, skewness, and kurtosis) as they are all undefined. Mode location (m) and scale (γ) are the only two parameters in this distribution. The location parameter specifies the peak or mode of the distribution while the scale parameter specifies the half-width at half-maximum of the distribution. In addition, the mean and variance of a cauchy or Lorentzian distribution are undefined. In addition, the cauchy distribution is the Student's t distribution with only 1 degree of freedom. This distribution is also constructed by taking the ratio of two standard normal distributions (normal distributions with a mean of zero and a variance of one) that are independent of one another. The input requirements are such that Location can be any value whereas Scale >0 and can be any positive value.

Chi-Square Distribution

The chi-square distribution is a probability distribution used predominatly in hypothesis testing, and is related to the gamma distribution and the standard normal distribution. For instance, the sums of independent normal distributions are distributed as a chi-square (χ²) with k degrees of freedom: Z₁²+Z₂²+ . . . +Zₖ²^{^{d}}˜χₖ²

The mathematical constructs for the chi-square distribution are as follows:

$$f ( x ) = \frac{{2}^{- k / 2}}{Γ ( k / 2 )} {x}^{k / 2 - 1} {e}^{- x / 2} for all x > 0$$

$$mean = k$$

$$standard deviation = \sqrt{2 k}$$

$$skewness = 2 \sqrt{\frac{2}{k}}$$

$$excess kurtosis = \frac{12}{k}$$

Γ is the gamma function. Degrees of freedom k is the only distributional parameter. The chi-square distribution can also be modeled using a gamma distribution by setting the shape

$$parameter = \frac{k}{2} and scale = 2 {S}^{2}$$

where S is the scale. The input requirements are such that Degrees of freedom >1 and must be an integer <1000.

Exponential Distribution

The exponential distribution is widely used to describe events recurring at random points in time, such as the time between failures of electronic equipment or the time between arrivals at a service booth. It is related to the Poisson distribution, which describes the number of occurrences of an event in a given interval of time. An important characteristic of the exponential distribution is the “memoryless” property, which means that the future lifetime of a given object has the same distribution, regardless of the time it existed. In other words, time has no effect on future outcomes. The mathematical constructs for the exponential distribution are as follows:

$$f ( x ) = λ {e}^{- λ x} for x \geq 0 ; λ > 0$$

$$mean = \frac{1}{λ}$$

$$standard deviation = \frac{1}{λ}$$

$$skewness = 2 ( this value applies to all success rate λ inputs )$$

$$excess kurtosis = 6 ( this value applies to all success rate λ inputs )$$

Success rate (λ) is the only distributional parameter. The number of successful trials is denoted x.

The condition underlying the exponential distribution is:
The exponential distribution describes the amount of time between occurrences.

Input requirements: Rate >0 and ≦300

Extreme Value Distribution or Gumbel Distribution

The extreme value distribution (Type 1) is commonly used to describe the largest value of a response over a period of time, for example, in flood flows, rainfall, and earthquakes. Other applications include the breaking strengths of materials, construction design, and aircraft loads and tolerances. The extreme value distribution is also known as the Gumbel distribution.

The mathematical constructs for the extreme value distribution are as follows:

$$f ( x ) = \frac{1}{β} z {e}^{- Z} where z = {e}^{\frac{x - m}{β}}$$

$$for β > 0 ; and any value of x and m$$

$$mean = m + 0.577215 β$$

$$standard deviation = \sqrt{\frac{1}{6} {π}^{2} {β}^{2}}$$

$$\begin{matrix}skewness = \frac{12 \sqrt{6} ( 1.2020569 )}{{π}^{3}} \\ = 1.13955 ( this applies for all values of mode and scale ) \end{matrix}$$

$$excess kurtosis = 5.4 ( this applies for all values of mode and scale )$$

Mode (m) and scale (β) are the distributional parameters. There are two standard parameters for the extreme value distribution: mode and scale. The mode parameter is the most likely value for the variable (the highest point on the probability distribution). The scale parameter is a number greater than 0. The larger the scale parameter, the greater the variance. The input requirements are such that Mode can be any value and Scale >0.

F Distribution or Fisher-Snedecor Distribution

The F distribution, also known as the Fisher-Snedecor distribution, is another continuous distribution used most frequently for hypothesis testing. Specifically, it is used to test the statistical difference between two variances in analysis of variance tests and likelihood ratio tests. The F distribution with the numerator degree of freedom n and denominator degree of freedom m is related to the chi-square distribution in that:

$$\frac{{χ}_{n}^{2} / {n}^{d}}{{χ}_{m}^{2} / m} ∼ {F}_{n , m}$$

$$or$$

$$f ( x ) = \frac{Γ ( \frac{n + m}{2} ) {( \frac{n}{m} )}^{n / 2} {x}^{n / 2 - 1}}{Γ ( \frac{n}{2} ) {Γ ( \frac{m}{2} ) [ x ( \frac{n}{m} ) + 1 ]}^{( n + m ) / 2}}$$

$$mean = \frac{m}{m - 2}$$

$$standard deviation = \frac{2 {m}^{2} ( m + n - 2 )}{{n ( m - 2 )}^{2} ( m - 4 )} for all m > 4$$

$$skewness = \frac{2 ( m + 2 n - 2 )}{m - 6} \sqrt{\frac{2 ( m - 4 )}{n ( m + n - 2 )}}$$

$$excess kurtosis = \frac{12 ( - 16 + 20 m - 8 {m}^{2} + {m}^{3} + 44 n - 32 mn + 5 {m}^{2} n - 22 {n}^{2} + 5 {mn}^{2}}{n ( m - 6 ) ( m - 8 ) ( n + m - 2 )}$$

The numerator degree of freedom n and denominator degree of freedom m are the only distributional parameters. The input requirements are such that Degrees of freedom numerator and degrees of freedom denominator both >0 integers.

Gamma Distribution (Erlang Distribution)

The gamma distribution applies to a wide range of physical quantities and is related to other distributions: lognormal, exponential, Pascal, Erlang, Poisson, and Chi-Square. It is used in meteorological processes to represent pollutant concentrations and precipitation quantities. The gamma distribution is also used to measure the time between the occurrence of events when the event process is not completely random. Other applications of the gamma distribution include inventory control, economic theory, and insurance risk theory.

The gamma distribution is most often used as the distribution of the amount of time until the rth occurrence of an event in a Poisson process. When used in this fashion, the three conditions underlying the gamma distribution are:
The number of possible occurrences in any unit of measurement is not limited to a fixed number.
The occurrences are independent. The number of occurrences in one unit of measurement does not affect the number of occurrences in other units.
The average number of occurrences must remain the same from unit to unit.

The mathematical constructs for the gamma distribution are as follows:

$$f ( x ) = \frac{{( \frac{x}{β} )}^{α - 1} {e}^{- \frac{x}{β}}}{Γ ( α ) β} with any value of α > 0 and β > 0$$

$$mean = α β$$

$$standard deviation = \sqrt{α {β}^{2}}$$

$$skewness = \frac{2}{\sqrt{α}}$$

$$excess kurtosis = \frac{6}{α}$$

Shape parameter alpha (α) and scale parameter beta (β) are the distributional parameters, and Γ is the gamma function. When the alpha parameter is a positive integer, the gamma distribution is called the Erlang distribution, used to predict waiting times in queuing systems, where the Erlang distribution is the sum of independent and identically distributed random variables each having a memoryless exponential distribution. Setting n as the number of these random variables, the mathematical construct of the Erlang distribution is:

$$f ( x ) = \frac{{x}^{n - 1} {e}^{- x}}{( n - 1 ) !}$$

for all x>0 and all positive integers of n, where the input requirements are such that Scale Beta >0 and can be any positive value, Shape Alpha ≧0.05 and any positive value, and Location can be any value.

Logistic Distribution

The logistic distribution is commonly used to describe growth, that is, the size of a population expressed as a function of a time variable. It also can be used to describe chemical reactions and the course of growth for a population or individual.

The mathematical constructs for the logistic distribution are as follows:

$$f ( x ) = \frac{{e}^{\frac{μ - x}{α}}}{{α [ 1 + {e}^{( \frac{μ - x}{α} )} ]}^{2}} for any value of α and μ$$

$$mean = μ$$

$$standard deviation = \sqrt{\frac{1}{3} {π}^{2} {α}^{2}}$$

$$skewness = 0 ( this applies to all mean and scale inputs )$$

$$excess kurtosis = 1.2 ( this applies to all mean and scale inputs )$$

Mean (μ) and scale (α) are the distributional parameters. There are two standard parameters for the logistic distribution: mean and scale. The mean parameter is the average value, which for this distribution is the same as the mode, because this distribution is symmetrical. The scale parameter is a number greater than 0. The larger the scale parameter, the greater the variance. Input requirements: Scale >0 and can be any positive value, Mean can be any value

Lognormal Distribution

The lognormal distribution is widely used in situations where values are positively skewed, for example, in financial analysis for security valuation or in real estate for property valuation, and where values cannot fall below zero. Stock prices are usually positively skewed rather than normally (symmetrically) distributed. Stock prices exhibit this trend because they cannot fall below the lower limit of zero but might increase to any price without limit. Similarly, real estate prices illustrate positive skewness and are lognormally distributed as property values cannot become negative.

The three conditions underlying the lognormal distribution are:
The uncertain variable can increase without limits but cannot fall below zero.
The uncertain variable is positively skewed, with most of the values near the lower limit.
The natural logarithm of the uncertain variable yields a normal distribution.

Generally, if the coefficient of variability is greater than 30 percent, use a lognormal distribution. Otherwise, use the normal distribution. The mathematical constructs for the lognormal distribution are as follows:

$$f ( x ) = \frac{1}{x \sqrt{2 π} ln ( σ )} {e}^{- \frac{{[ ln ( x ) - ln ( μ ) ]}^{2}}{{2 [ ln ( σ ) ]}^{2}}}$$

$$for x > 0 ; μ > 0 and σ > 0$$

$$mean = exp ( μ + \frac{{σ}^{2}}{2} )$$

$$standard deviation = \sqrt{exp ( {σ}^{2} + 2 μ ) [ exp ( {σ}^{2} ) - 1 ]}$$

$$skewness = ⌊ \sqrt{exp ( {σ}^{2} ) - 1} ⌋ ( 2 + exp ( {σ}^{2} ) )$$

$$excess kurtosis = exp ( 4 {σ}^{2} ) + 2 exp ( 3 {σ}^{2} ) + 3 exp ( 2 {σ}^{2} ) - 6$$

Mean (μ) and standard deviation (σ) are the distributional parameters. The input requirements are such that Mean and Standard deviation are both >0 and can be any positive value. By default, the lognormal distribution uses the arithmetic mean and standard deviation. For applications for which historical data are available, it is more appropriate to use either the logarithmic mean and standard deviation, or the geometric mean and standard deviation.

Normal Distribution

The normal distribution is the most important distribution in probability theory because it describes many natural phenomena, such as people's IQs or heights. Decision makers can use the normal distribution to describe uncertain variables such as the inflation rate or the future price of gasoline.

Conditions

The three conditions underlying the normal distribution are:
Some value of the uncertain variable is the most likely (the mean of the distribution).
The uncertain variable could as likely be above the mean as it could be below the mean (symmetrical about the mean).
The uncertain variable is more likely to be in the vicinity of the mean than further away.

The mathematical constructs for the normal distribution are as follows:

$$f ( x ) = \frac{1}{\sqrt{2 π} σ} {e}^{- \frac{{( x - μ )}^{2}}{2 {σ}^{2}}} for all values of x and μ ;$$

$$while σ > 0$$

$$mean = μ$$

$$standard deviation = σ \\ skewness = 0 ( this applies to all inputs of mean and standard deviation )$$

$$excess kurtosis = 0 ( this applies to all inputs of mean and standard deviation )$$

Mean (μ) and standard deviation (σ) are the distributional parameters. The input requirements are such that Standard deviation >0 and can be any positive value and Mean can be any value.

Pareto Distribution

The Pareto distribution is widely used for the investigation of distributions associated with such empirical phenomena as city population sizes, the occurrence of natural resources, the size of companies, personal incomes, stock price fluctuations, and error clustering in communication circuits.

The mathematical constructs for the pareto are as follows:

$$f ( x ) = \frac{β {L}^{β}}{{x}^{( 1 + β )}} for x > L$$

$$mean = \frac{β L}{β - 1}$$

$$standard deviation = \sqrt{\frac{β {L}^{2}}{{( β - 1 )}^{2} ( β - 2 )}}$$

$$skewness = \sqrt{\frac{β - 2}{β}} [ \frac{2 ( β + 1 )}{β - 3} ]$$

$$excess kurtosis = \frac{6 ( {β}^{3} + {β}^{2} - 6 β - 2 )}{β ( β - 3 ) ( β - 4 )}$$

Location (L) and shape (β) are the distributional parameters.

There are two standard parameters for the Pareto distribution: location and shape. The location parameter is the lower bound for the variable. After you select the location parameter, you can estimate the shape parameter. The shape parameter is a number greater than 0, usually greater than 1. The larger the shape parameter, the smaller the variance and the thicker the right tail of the distribution. The input requirements are such that Location >0 and can be any positive value while Shape ≧0.05.

Student's t Distribution

The Student's t distribution is the most widely used distribution in hypothesis test. This distribution is used to estimate the mean of a normally distributed population when the sample size is small, and is used to test the statistical significance of the difference between two sample means or confidence intervals for small sample sizes.

$$f ( t ) = \frac{Γ [ ( r + 1 ) / 2 ]}{\sqrt{r π} Γ [ r / 2 ]} {( 1 + {t}^{2} / r )}^{- ( r + 1 ) / 2}$$

$$mean = 0 ( this applies to all degrees of freedom r except if the distribution is shifted to another nonzero central location )$$

$$standard deviation = \sqrt{\frac{r}{r - 2}}$$

$$skewness = 0$$

$$excess kurtosis = \frac{6}{r - 4} for all r > 4$$

$$where t = \frac{x - \overline{x}}{s} and Γ is the gamma function .$$

Degree of freedom r is the only distributional parameter. The t-distribution is related to the F-distribution as follows: the square of a value of t with r degrees of freedom is distributed as F with 1 and r degrees of freedom. The overall shape of the probability density function of the t-distribution also resembles the bell shape of a normally distributed variable with mean 0 and variance 1, except that it is a bit lower and wider or is leptokurtic (fat tails at the ends and peaked center). As the number of degrees of freedom grows (say, above 30), the t-distribution approaches the normal distribution with mean 0 and variance 1. The input requirements are such that Degrees of freedom ≧1 and must be an integer.

Triangular Distribution

The triangular distribution describes a situation where you know the minimum, maximum, and most likely values to occur. For example, you could describe the number of cars sold per week when past sales show the minimum, maximum, and usual number of cars sold.

Conditions

The three conditions underlying the triangular distribution are:
The minimum number of items is fixed.
The maximum number of items is fixed.
The most likely number of items falls between the minimum and maximum values, forming a triangular-shaped distribution, which shows that values near the minimum and maximum are less likely to occur than those near the most-likely value.

The mathematical constructs for the triangular distribution are as follows:

$$f ( x ) = \{ \begin{matrix}\frac{2 ( x - Min )}{( Max - Min ) ( Likely - min )} & for Min < x < Likely \\ \frac{2 ( Max - x )}{( Max - Min ) ( Max - Likely )} & for Likely < x < Max\end{matrix} \\ mean = \frac{1}{3} ( Min + Likely + Max ) \\ standard deviation = \sqrt{\frac{1}{18} ( {Min}^{2} + {Likely}^{2} + {Max}^{2} - Min Max - Min Likely - Max Likely )} \\ skewness = \frac{\begin{matrix}\sqrt{2} ( Min + Max - 2 Likely ) \\ ( 2 Min - Max - Likely ) ( Min - 2 Max + Likely )\end{matrix}}{5 {( \begin{matrix}{Min}^{2} + {Max}^{2} + {Likely}^{2} - MinMax - \\ Min Likely - Max Likely\end{matrix} )}^{3 / 2}} \\ excess kurtosis = - 0.6$$

Minimum (Min), most likely (Likely) and maximum (Max) are the distributional parameters and the input requirements are such that Min≦Most Likely≦Max and can take any value, Min<Max and can take any value.

Uniform Distribution

With the uniform distribution, all values fall between the minimum and maximum and occur with equal likelihood.

The three conditions underlying the uniform distribution are:
The minimum value is fixed.
The maximum value is fixed.
All values between the minimum and maximum occur with equal likelihood.

The mathematical constructs for the uniform distribution are as follows:

$$f ( x ) = \frac{1}{Max - Min}$$

$$for all values such that Min < Max$$

$$mean = \frac{Min + Max}{2}$$

$$standard deviation = \sqrt{\frac{{( Max - Min )}^{2}}{12}}$$

$$skewness = 0$$

$$excess kurtosis = - 1.2$$

$$( this applies to all inputs of Min and Max )$$

Maximum value (Max) and minimum value (Min) are the distributional parameters. The input requirements are such that Min<Max and can take any value.

Weibull Distribution (Rayleigh Distribution)

The Weibull distribution describes data resulting from life and fatigue tests. It is commonly used to describe failure time in reliability studies as well as the breaking strengths of materials in reliability and quality control tests. Weibull distributions are also used to represent various physical quantities, such as wind speed. The Weibull distribution is a family of distributions that can assume the properties of several other distributions. For example, depending on the shape parameter you define, the Weibull distribution can be used to model the exponential and Rayleigh distributions, among others. The Weibull distribution is very flexible. When the Weibull shape parameter is equal to 1.0, the Weibull distribution is identical to the exponential distribution. The Weibull location parameter lets you set up an exponential distribution to start at a location other than 0.0. When the shape parameter is less than 1.0, the Weibull distribution becomes a steeply declining curve. A manufacturer might find this effect useful in describing part failures during a burn-in period.

The mathematical constructs for the Weibull distribution are as follows:

$$f ( x ) = {\frac{α}{β} [ \frac{x}{β} ]}^{α - 1} {e}^{- {( \frac{x}{β} )}^{α}}$$

$$mean = β Γ ( 1 + {α}^{- 1} )$$

$$standard deviation = {β}^{2} [ Γ ( 1 + 2 {α}^{- 1} ) - {Γ}^{2} ( 1 + {α}^{- 1} ) ]$$

$$skewness = \frac{2 {Γ}^{3} ( 1 + {β}^{- 1} ) - 3 Γ ( 1 + {β}^{- 1} ) Γ ( 1 + 2 {β}^{- 1} ) + Γ ( 1 + 3 {β}^{- 1} )}{{[ Γ ( 1 + 2 {β}^{- 1} ) - {Γ}^{2} ( 1 + {β}^{- 1} ) ]}^{3 / 2}}$$

$$excess kurtosis = \frac{\begin{matrix}- 6 {Γ}^{4} ( 1 + {β}^{- 1} ) + 12 {Γ}^{2} ( 1 + {β}^{- 1} ) Γ ( 1 + 2 {β}^{- 1} ) - \\ 3 {Γ}^{2} ( 1 + 2 {β}^{- 1} ) - 4 Γ ( 1 + {β}^{- 1} ) Γ ( 1 + 3 {β}^{- 1} ) + Γ ( 1 + 4 {β}^{- 1} )\end{matrix}}{{[ Γ ( 1 + 2 {β}^{- 1} ) - {Γ}^{2} ( 1 + {β}^{- 1} ) ]}^{2}}$$

Location (L), shape (α) and scale (β) are the distributional parameters, and Γ is the Gamma function. The input requirements are such that Scale >0 and can be any positive value, Shape ≧0.05 and Location can take on any value.

Multiple Regression Analysis and Econometric Data Analysis

This section demonstrates the mathematical models and computations used in creating the general regression equations, which take the form of Y=β₀+β₁X₁+β₂X₂+ . . . +βₙXₙ+ε where β₀ is the intercept, β₁ are the slope coefficients, and ε is the error term. The Y term is the dependent variable and the X terms are the independent variables, where these X variables are also known as the regressors. The dependent variable is named as such as it depends on the independent variable, for example, sales revenue depends on the amount of marketing costs expended on a product's advertising and promotion, making the dependent variable sales and the independent variable marketing costs. An example of a bivariate regression where there is only a single Y and a single X variable, is seen as simply inserting the best-fitting line through a set of data points in a two-dimensional plane. In other cases, a multivariate regression can be performed, where there are multiple or k number of independent X variables or regressors where in this case, the best-fitting line will be within a k+1 dimensional plane.

Fitting a line through a set of data points in a multidimensional scatter plot may result in numerous possible lines. The best-fitting line is defined as the single unique line that minimizes the total vertical errors, that is, the sum of the absolute distances between the actual data points (Yᵢ) and the estimated line ( [symbol] ). To find the best-fitting unique line that minimizes the errors, a more sophisticated approach is applied, using multivariate regression analysis. Regression analysis therefore finds the unique best-fitting line by requiring that the total errors be minimized, or by calculating:

$$Min {\sum }_{i = 1}^{n} {( {Y}_{i} - {\hat{Y}}_{i} )}^{2}$$

Only one unique line will minimize this sum of squared errors as shown in the equation above. The errors (vertical distances between the actual data and the predicted line) are squared to avoid the negative errors from canceling out the positive errors. Solving this minimization problem with respect to the slope and intercept requires calculating first derivatives and setting them equal to zero:

$$\frac{d}{d {β}_{0}} {\sum }_{i = 1}^{n} {( {Y}_{i} - {\hat{Y}}_{i} )}^{2} = 0 and$$

$$\frac{d}{d {β}_{1}} {\sum }_{i = 1}^{n} {( {Y}_{i} - {\hat{Y}}_{i} )}^{2} = 0$$

Which yields the simple bivariate regression's set of least squares equations:

$${β}_{1} = \frac{{\sum }_{i = 1}^{n} ( {X}_{i} - \overline{X} ) ( {Y}_{i} - \overline{Y} )}{{\sum }_{i = 1}^{n} {( {X}_{i} - \overline{X} )}^{2}} = \frac{{\sum }_{i = 1}^{n} {X}_{i} {Y}_{i} - \frac{{\sum }_{i = 1}^{n} {X}_{i} {\sum }_{i = 1}^{n} {Y}_{i}}{n}}{{\sum }_{i = 1}^{n} {X}_{i}^{2} - \frac{{( {\sum }_{i = 1}^{n} {X}_{i} )}^{2}}{n}} \\ {β}_{0} = \overline{Y} - {β}_{1} \overline{X} \qquad$$

For multivariate regression, the analogy is expanded to account for multiple independent variables, where Yᵢ=β₁+β₂X_{2,i}+β₃X_{3,i}+εᵢ and the estimated slopes can be calculated by:

$${\hat{β}}_{2} = \frac{\sum {Y}_{i} {X}_{2 , i} \sum {X}_{3 , i}^{2} - \sum {Y}_{i} {X}_{3 , i} \sum {X}_{2 , i} {X}_{3 , i}}{\sum {X}_{2 , i}^{2} \sum {X}_{3 , i}^{2} - {( \sum {X}_{2 , i} {X}_{3 , i} )}^{2}}$$

$${\hat{β}}_{3} = \frac{\sum {Y}_{i} {X}_{3 , i} \sum {X}_{2 , i}^{2} - \sum {Y}_{i} {X}_{2 , i} \sum {X}_{2 , i} {X}_{3 , i}}{\sum {X}_{2 , i}^{2} \sum {X}_{3 , i}^{2} - {( \sum {X}_{2 , i} {X}_{3 , i} )}^{2}}$$

This set of results can be summarized using matrix notations: [X′X]⁻¹[X′Y].

In running multivariate regressions, great care must be taken to set up and interpret the results. For instance, a good understanding of econometric modeling is required (e.g., identifying regression pitfalls such as structural breaks, multicollinearity, heteroskedasticity, autocorrelation, specification tests, nonlinearities, and so forth) before a proper model can be constructed. Therefore the present invention includes some advanced econometrics approaches that are based on the principles of multiple regression outlined above.

One approach used is that of an Auto-ARIMA, which is based on the fundamental concepts of ARIMA theory or Autoregressive Integrated Moving Average models. ARIMA(p,d,q) models are the extension of the AR model that uses three components for modeling the serial correlation in the time series data. The first component is the autoregressive (AR) term. The AR(p) model uses the p lags of the time series in the equation. An AR(p) model has the form: yₜ=a₁yₜ₋₁+ . . . +aₚyₜ₋ₚ+eₜ. The second component is the integration (d) order term. Each integration order corresponds to differencing the time series. I(1) means differencing the data once. I(d) means differencing the data d times. The third component is the moving average (MA) term. The MA(q) model uses the q lags of the forecast errors to improve the forecast. An MA(q) model has the form: yₜ=eₜ+b₁eₜ₋₁+ . . . +b_{q}e_{t-q}. Finally, an ARMA(p,q) model has the combined form: yₜ=a₁yₜ₋₁+ . . . +aₚyₜ₋ₚ+eₜ+b₁eₜ₋₁+ . . . +b_{q}e_{t-q}. Using this ARIMA concept, various combinations of p, d, q integers are tested in an automated and systematic fashion to determine the best-fitting model for the user's data.

In order to determine the best fitting model, we apply several goodness-of-fit statistics to provide a glimpse into the accuracy and reliability of the estimated regression model. They usually take the form of a t-statistic, F-statistic, R-squared statistic, adjusted R-squared statistic, Durbin-Watson statistic, Akaike Criterion, Schwarz Criterion, and their respective probabilities.

The R-squared (R²), or coefficient of determination, is an error measurement that looks at the percent variation of the dependent variable that can be explained by the variation in the independent variable for a regression analysis. The coefficient of determination can be calculated by:

$${R}^{2} = 1 - \frac{{\sum }_{i = 1}^{n} {( {Y}_{i} - )}^{2}}{{\sum }_{i = 1}^{n} {( {Y}_{i} - \overline{Y} )}^{2}} = 1 - \frac{S S E}{T S S}$$

Where the coefficient of determination is one less the ratio of the sums of squares of the errors (SSE) to the total sums of squares (TSS). In other words, the ratio of SSE to TSS is the unexplained portion of the analysis, thus, one less the ratio of SSE to TSS is the explained portion of the regression analysis.

The estimated regression line is characterized by a series of predicted values ( [symbol] ); the average value of the dependent variable's data points is denoted Y̅; and the individual data points are characterized by Yᵢ. Therefore, the total sum of squares, that is, the total variation in the data or the total variation about the average dependent value, is the total of the difference between the individual dependent values and its average (the total squared distance of Yᵢ− Y̅). The explained sum of squares, the portion that is captured by the regression analysis, is the total of the difference between the regression's predicted value and the average dependent variable's data set (seen as the total squared distance of Ŷ− Y̅). The difference between the total variation (TSS) and the explained variation (ESS) is the unexplained sums of squares, also known as the sums of squares of the errors (SSE).

Another related statistic, the adjusted coefficient of determination, or the adjusted R-squared ( R̅²), corrects for the number of independent variables (k) in a multivariate regression through a degrees of freedom correction to provide a more conservative estimate:

$${\overline{R}}^{2} = 1 - \frac{{\sum }_{i = 1}^{n} {( {Y}_{i} - )}^{2} / ( k - 2 )}{{\sum }_{i = 1}^{n} {( {Y}_{i} - \overline{Y} )}^{2} / ( k - 1 )} = 1 - \frac{S S E / ( k - 2 )}{T S S / ( k - 1 )}$$

The adjusted R-squared should be used instead of the regular R-squared in multivariate regressions because every time an independent variable is added into the regression analysis, the R-squared will increase; indicating that the percent variation explained has increased. This increase occurs even when nonsensical regressors are added. The adjusted R-squared takes the added regressors into account and penalizes the regression accordingly, providing a much better estimate of a regression model's goodness-of-fit.

Other goodness-of-fit statistics include the t-statistic and the F-statistic. The former is used to test if each of the estimated slope and intercept(s) is statistically significant, that is, if it is statistically significantly different from zero (therefore making sure that the intercept and slope estimates are statistically valid). The latter applies the same concepts but simultaneously for the entire regression equation including the intercept and slopes. Using the previous example, the following illustrates how the t-statistic and F-statistic can be used in a regression analysis.

When running the Autoeconometrics methodology, multiple regression issues and errors are first tested for. These include items such as heteroskedasticity, multicollinearity, micronumerosity, lags, leads, autocorrelation and others. For instance, several tests exist to test for the presence of heteroskedasticity. These tests also are applicable for testing misspecifications and nonlinearities. The simplest approach is to graphically represent each independent variable against the dependent variable as illustrated earlier. Another approach is to apply one of the most widely used model, the White's test, where the test is based on the null hypothesis of no heteroskedasticity against an alternate hypothesis of heteroskedasticity of some unknown general form. The test statistic is computed by an auxiliary or secondary regression, where the squared residuals or errors from the first regression are regressed on all possible (and nonredundant) cross products of the regressors. For example, suppose the following regression is estimated:

Y=β₀+β₁X+β₂Z+εₜ

The test statistic is then based on the auxiliary regression of the errors (ε):

Eₜ²=α₀+α₁X+α₂Z+α₃X²+α₄Z²+α₅XZ+vₜ

The nR² statistic is the White's test statistic, computed as the number of observations (n) times the centered R-squared from the test regression. White's test statistic is asymptotically distributed as a χ² with degrees of freedom equal to the number of independent variables (excluding the constant) in the test regression.

The White's test is also a general test for model misspecification, because the null hypothesis underlying the test assumes that the errors are both homoskedastic and independent of the regressors, and that the linear specification of the model is correct. Failure of any one of these conditions could lead to a significant test statistic. Conversely, a nonsignificant test statistic implies that none of the three conditions is violated. For instance, the resulting F-statistic is an omitted variable test for the joint significance of all cross products, excluding the constant.

One method to fix heteroskedasticity is to make it homoskedastic by using a weighted least squares (WLS) approach. For instance, suppose the following is the original regression equation:

Y=β₀+β₁X₁+β₂X₂+β₃X₃+ε
Further suppose that X₂ is heteroskedastic. Then transform the data used in the regression into:

$$Y = \frac{{β}_{0}}{{X}_{2} } + {β}_{1} \frac{{X}_{1}}{{X}_{2}} + {β}_{2} + {β}_{3} \frac{{X}_{3}}{{X}_{2}} + \frac{ɛ}{{X}_{2}}$$

The model can be redefined as the following WLS regression:

Y_{WLS}=β₀^{WLS}+β₁^{WLS}X₁+β₂^{WLS}X₂+β₃^{WLS}X₃+v

Alternatively, the Park's test can be applied to test for heteroskedasticity and to fix it. The Park's test model is based on the original regression equation, uses its errors, and creates an auxiliary regression that takes the form of:

ln eₜ²=β₁+β₂ ln X_{k,t}

Suppose β₂ is found to be statistically significant based on a t-test, then heteroskedasticity is found to be present in the variable X_{k,i}. The remedy therefore is to use the following regression specification:

$$\frac{Y}{\sqrt{{X}_{k}^{{B}_{2}}} } = \frac{{β}_{1}}{\sqrt{{X}_{k}^{{β}_{2}}}} + \frac{{β}_{2} {X}_{2}}{\sqrt{{X}_{k}^{{β}_{2}}}} + \frac{{β}_{3} {X}_{3}}{\sqrt{{X}_{k}^{{β}_{2}}}} + ɛ .$$

Multicollinearity exists when there is a linear relationship between the independent variables. When this occurs, the regression equation cannot be estimated at all. In near collinearity situations, the estimated regression equation will be biased and provide inaccurate results. This situation is especially true when a step-wise regression approach is used, where the statistically significant independent variables will be thrown out of the regression mix earlier than expected, resulting in a regression equation that is neither efficient nor accurate.

As an example, suppose the following multiple regression analysis exists, where:

Yᵢ=β₁+β₂X_{2,i}+β₃X_{3,i}+εᵢ

The estimated slopes can be calculated through:

$${\hat{β}}_{2} = \frac{\sum {Y}_{i} {X}_{2 , i} \sum {X}_{3 , i}^{2} - \sum {Y}_{i} {X}_{3 , i} \sum {X}_{2 , i} {X}_{3 , i}}{\sum {X}_{2 , i}^{2} \sum {X}_{3 , i}^{2} - {( \sum {X}_{2 , i} {X}_{3 , i} )}^{2}}$$

$${\hat{β}}_{3} = \frac{\sum {Y}_{i} {X}_{3 , i} \sum {X}_{2 , i}^{2} - \sum {Y}_{i} {X}_{2 , i} \sum {X}_{2 , i} {X}_{3 , i}}{\sum {X}_{2 , i}^{2} \sum {X}_{3 , i}^{2} - {( \sum {X}_{2 , i} {X}_{3 , i} )}^{2}}$$

Now suppose that there is perfect multicollinearity, that is, there exists a perfect linear relationship between X₂ and X₃, such that X_{3,i}=λX_{2,i} for all positive values of λ. Substituting this linear relationship into the slope calculations for β₂, the result is indeterminate. In other words, we have:

$${\hat{β}}_{2} = \frac{\sum {Y}_{i} {X}_{2 , i} \sum {λ}^{2} {X}_{2 , i}^{2} - \sum {Y}_{i} λ {X}_{2 , i} \sum λ {X}_{2 , i}^{2}}{\sum {X}_{2 , i}^{2} \sum {λ}^{2} {X}_{2 , i}^{2} - {( \sum λ {X}_{2 , i}^{2} )}^{2}} = \frac{0}{0}$$

The same calculation and results apply to β₃, which means that the multiple regression analysis breaks down and cannot be estimated given a perfect collinearity condition.

One quick test of the presence of multicollinearity in a multiple regression equation is that the R-squared value is relatively high while the t-statistics are relatively low. Another quick test is to create a correlation matrix between the independent variables. A high cross correlation indicates a potential for multicollinearity. The rule of thumb is that a correlation with an absolute value greater than 0.75 is indicative of severe multicollinearity.
Another test for multicollinearity is the use of the variance inflation factor (VIF), obtained by regressing each independent variable to all the other independent variables, obtaining the R-squared value and calculating the VIF of that variable by estimating:

$$V I {F}_{i} = \frac{1}{( 1 - {R}_{i}^{2} )}$$

A high VIF value indicates a high R-squared near unity. As a rule of thumb, a VIF value greater than 10 is usually indicative of destructive multicollinearity. The Autoeconometrics method computes for multicollinearity and corrects the data before running the next iteration when enumerating through the entire set of possible combinations and permutations of models.

One very simple approach to test for autocorrelation is to graph the time series of a regression equation's residuals. If these residuals exhibit some cyclicality, then autocorrelation exists. Another more robust approach to detect autocorrelation is the use of the Durbin-Watson statistic, which estimates the potential for a first-order autocorrelation. The Durbin-Watson test also identifies model misspecification. That is, if a particular time-series variable is correlated to itself one period prior. Many time-series data tend to be autocorrelated to their historical occurrences. This relationship can be due to multiple reasons, including the variables' spatial relationships (similar time and space), prolonged economic shocks and events, psychological inertia, smoothing, seasonal adjustments of the data, and so forth.

The Durbin-Watson statistic is estimated by the sum of the squares of the regression errors for one period prior, to the sum of the current period's errors:

$$DW = \frac{\sum {( {ɛ}_{t} - {ɛ}_{t - 1} )}^{2}}{\sum {ɛ}_{t}^{2}}$$

Another test for autocorrelation is the Breusch-Godfrey test, where for a regression function in the form of:

Y=ƒ(X₁,X₂, . . . ,Xₖ)

Estimate this regression equation and obtain its errors εₜ. Then, run the secondary regression function in the form of:

Y=ƒ(X₁,X₂, . . . ,Xₖ,εₜ₋₁,εₜ₋₂,εₜ₋ₚ)

Obtain the R-squared value and test it against a null hypothesis of no autocorrelation versus an alternate hypothesis of autocorrelation, where the test statistic follows a Chi-Square distribution of p degrees of freedom:

R²(n−p)˜χ_{df=p}²

Fixing autocorrelation requires the application of advanced econometric models including the applications of ARIMA (as described above) or ECM (Error Correction Models). However, one simple fix is to take the lags of the dependent variable for the appropriate periods, add them into the regression function, and test for their significance, for instance:

Yₜ=ƒ(Yₜ₋₁,Yₜ₋₂, . . . ,Yₜ₋ₚ,X₁,X₂, . . . ,Xₖ)

In interpreting the results of an Autoeconometrics model, most of the specifications are identical to the multivariate regression analysis. However, there are several additional sets of results specific to the econometric analysis. The first is the addition of Akaike Information Criterion (AIC) and Schwarz Criterion (SC), which are often used in ARIMA model selection and identification. That is, AIC and SC are used to determine if a particular model with a specific set of p, d, and q parameters is a good statistical fit. SC imposes a greater penalty for additional coefficients than the AIC but generally, the model with the lowest AIC and SC values should be chosen. Finally, an additional set of results called the autocorrelation (AC) and partial autocorrelation (PAC) statistics are provided in the ARIMA report.

For instance, if autocorrelation AC(1) is nonzero, it means that the series is first order serially correlated. If AC dies off more or less geometrically with increasing lags, it implies that the series follows a low-order autoregressive process. If AC drops to zero after a small number of lags, it implies that the series follows a low-order moving-average process. In contrast, PAC measures the correlation of values that are k periods apart after removing the correlation from the intervening lags. If the pattern of autocorrelation can be captured by an autoregression of order less than k, then the partial autocorrelation at lag k will be close to zero. The Ljung-Box Q-statistics and their p-values at lag k are also provided, where the null hypothesis being tested is such that there is no autocorrelation up to order k. The dotted lines in the plots of the autocorrelations are the approximate two standard error bounds. If the autocorrelation is within these bounds, it is not significantly different from zero at approximately the 5% significance level. Finding the right ARIMA model takes practice and experience. These AC, PAC, SC, and AIC are highly useful diagnostic tools to help identify the correct model specification. Finally, the ARIMA parameter results are obtained using sophisticated optimization and iterative algorithms, which means that although the functional forms look like those of a multivariate regression, they are not the same. ARIMA is a much more computationally intensive and advanced econometric approach.

Description of Global Application and Contingencies

The present invention applies to the domestic health-care marketplace in the United States with extraterritorial applications across national and international boundaries. Other countries have looked to the United States as a leader in health-care innovation and have adopted many of the inventions with respect to health-care infrastructure and financing. An example of such an adoption is that of the diagnosis-related groups, or DRGs. In the early 1960s researchers at Yale University developed DRGs as a reimbursement methodology that aligned a hospital's workload to its costs, at an individual level (case-by-case) and by hospital (global level). In 1983 Medicare adopted the DRG-based scheme as a part of a prospective payment system for hospital inpatient treatments. In the mid-1980s commercial health plans in the United States adopted the DRG methodology as part of their provider contracting payment system for inpatient services with hospitals. In 1992 Australia, in 2002 Germany, and in 2008 Switzerland each adopted a DRG-based system. The present invention is designed for comparable adoption, adaptation, and customization across borders.

The present invention's application to the domestic health-care marketplace in the United States is based on the continuation of the Affordable Care Act as passed in its entirety. The changes could be dramatic with a change in the Presidency, Senate, and House as well as the Supreme Court's judicial review of the health-care legislation. Notwithstanding, this present invention was designed to pivot and accommodate for the contingencies that may emerge as a result of these consequences. We have anticipated the following changes to the legislation as possibilities: elimination of the individual mandate, discontinuation in the development of the state-run Health Insurance Exchanges, abolition of premium tax credits, rejection of employer-based premium tax penalties, dismissal of the expanded eligibility requirement, repeal of the medical loss ratio requirements, and a reversal of the essential health benefits coverage requirement. Similarly, we have also anticipated the following as a result of the repeal of the existing legislation: the acceptance of privately run health insurance exchanges, the growth of defined contribution health plans, the elimination of statutory cross-border insurance barriers to entry, abolition of the tax-favored status of employer-based health insurance, and the detachment of employment-based insurance coverage. The present invention has no impact on simulation, optimization, cohort analysis, and time-series forecasting. Where it does have impact is in the area of real options analysis. However, the present invention is capable of accepting the tweaks and modifications necessary to never be in peril of obsolescence as discussed below.

Real options are not limited to the compulsory requirements driven by legislative action, but may emerge as a result of enabling legislation and coexist within a whole portfolio of possibilities. For example, adopting a defined health-care contribution approach is an option that may be considered a solution that is independent of health reform legislation where a corporation may decide to set a contribution amount and shift the purchasing decision of health-care coverage to the employee for purchase in the open market. Another example is a decision by a corporation to sponsor a high-deductible health plan where it elects to either fund or not fund health savings accounts. A third example is an option resulting from the enabling health reform legislation where the corporation may elect to terminate employer-sponsored coverage and pay a penalty. If the legislation is repealed and the penalty is no longer required, this last option is no longer valid and is deleted from the portfolio; however, the portfolio is far from depleted and retains all of the other options available to the corporation.

The development of the health insurance exchanges in each of the individual states as required by the health-care legislation was conceived to provide individuals and small businesses an opportunity to purchase health care with group buying power. The federal government also has a role in that if a state decides not to build an exchange, the federal government will step in to perform this function, and that the Office of Personnel Management (OPM) must provide two multistate qualified health plan options in each individual state's insurance exchange. In the event that this legislation is repealed, the legislation is defunded, or agencies are directed to cease and desist with guideline issuance, the state-run exchange development will fail. The implications are broad in that the market, as it exists today, would be virtually unchanged and the advent of premium tax credits and cost sharing subsidies would never take effect. Notwithstanding, it is quite possible that private health insurance exchanges may get revitalized and emerge as a market alternative contributing as a real option within the portfolio under a scenario where this legislation is repealed.

The individual mandate is the requirement that each person must either be insured for essential health benefits coverage or pay a penalty. If this requirement is deemed unconstitutional, then the implications are that the forced purchase of health insurance coverage will significantly impact the funding of the legislation, may reduce the number of individuals deciding to review options for coverage, and would eliminate the penalty tax under an options analysis. As with the corporate real options result, if the legislation is repealed and the penalty is no longer required, the penalty option is no longer valid and is deleted from the portfolio, but all of the other options are retained and available for the individual.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special-purpose hardware and computer instructions; by combinations of general-purpose hardware and computer instructions; and so on—any and all of which may be generally referred to herein as a “circuit,” “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more substeps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

Traditionally, a computer program consists of a finite sequence of computational instructions or program instructions. It will be appreciated that a programmable apparatus (i.e., computing device) can receive such a computer program and, by processing the computational instructions thereof, produce a further technical effect.

A programmable apparatus includes one or more microprocessors, microcontrollers, embedded microcontrollers, programmable digital signal processors, programmable devices, programmable gate arrays, programmable array logic, memory devices, application-specific integrated circuits, or the like that can be suitably employed or configured to process computer program instructions, execute computer logic, store computer data, and so on. Throughout this disclosure and elsewhere a computer can include any and all suitable combinations of at least one general-purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer-readable medium(s) may be utilized. The computer-readable medium may be a computer-readable signal medium or a computer-readable storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device.

Program code embodied on a computer-readable medium may be transmitted using any appropriate medium, including but not limited to wireless, wireline, optical fiber cable, RF, and so forth, or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

In view of the foregoing, it will now be appreciated that elements of the block diagrams and flowchart illustrations support combinations of means for performing the specified functions, combinations of steps for performing the specified functions, program instruction means for performing the specified functions, and so on.

It will be appreciated that computer program instructions may include computer executable code. A variety of languages for expressing computer program instructions are possible, including without limitation C, C++, Java, JavaScript, assembly language, Lisp, and so on. Such languages may include assembly languages, hardware description languages, database programming languages, functional programming languages, imperative programming languages, and so on. In some embodiments, computer program instructions can be stored, compiled, or interpreted to run on a computer, a programmable data processing apparatus, a heterogeneous combination of processors or processor architectures, and so on. Without limitation, embodiments of the system as described herein can take the form of web-based computer software, which includes client/server software, software-as-a-service, peer-to-peer software, or the like.

In some embodiments, a computer enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computer can process these threads based on priority or any other order based on instructions provided in the program code.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

1. A programmed computer system comprising:
a health quant data modeler (HQDM) module comprising physical memory storing instructions that cause the HQDM module to:
provide a user interface to a user via said HQDM module;
create a user profile, wherein said user profile stores all input assumptions entered by said user;
provide said user with a set of analytical models;
receive from said user a model selection, wherein said model selection is selected from said set of analytical models;
request input parameters from said user;
receive said input parameters from said user; wherein one or more of said input parameters are selected from a group of input parameters comprising existing data files, manual inputs, data model fitting, simulation assumptions, and Structured Query Language (SQL) expressions;
process said input parameters, wherein said input parameters are mapped based on said model selection;
provide said user analysis customization options, wherein said analysis customization options are comprised of analysis parameters, analysis variables, and analysis options;
receive from said user analysis customization input, wherein said user selects and customizes one or more of said analysis customization options;
analyze mapped input parameters, wherein said mapped input parameters are analyzed based on said analysis customization input;
generate one or more reports, wherein said one or more reports are based on the analysis of said mapped input parameters;
provide said user said one or more reports; wherein said one or more reports are selected from a group of reports comprising, charts, graphs, data tables, and text files.

2. The programmed computer system of claim 1, wherein said HQDM is used for healthcare finance decision analysis.

3. The programmed computer system of claim 2, wherein said HQDM is used as an employer-sponsored health care strategy and finance decision support utility that integrates one or more decision factors selected from a group of decision factors comprising i) insurance based design and funding methods, ii) non-insurance based design and funding methods, iii) corporate income tax, iv) individual income tax, v) adjustments in employee compensation, vi) structures in account based funding, vii) organizational considerations in types of employment reconfigurations, viii) eligibility determinations, and ix) percentage of income calculations for individual premium tax credits and penalties.

4. The programmed computer system of claim 1 further comprising a database communicatively linked to said HQDM module.

5. The programmed computer system of claim 1, wherein said HQDM module is further comprised of physical memory storing instructions that cause the HQDM module to extract said one or more reports to an external software program.

6. The programmed computer system of claim 1, wherein said HQDM module is further comprised of physical memory storing instructions that cause the HQDM module to store said one or more reports for future use as said input parameters.

7. A computerized method for healthcare decision analysis, the method comprising the steps of:
providing a user interface to a user via said HQDM module;
creating a user profile, wherein said user profile stores all input assumptions entered by said user;
providing said user with a set of analytical models;
receiving from said user a model selection, wherein said model selection is selected from said set of analytical models;
requesting input parameters from said user;
receiving said input parameters from said user; wherein one or more of said input parameters are selected from a group of input parameters comprising existing data files, manual inputs, data model fitting, simulation assumptions, and Structured Query Language (SQL) expressions;
processing said input parameters, wherein said input parameters are mapped based on said model selection;
providing said user analysis customization options, wherein said analysis customization options are comprised of analysis parameters, analysis variables, and analysis options;
receiving from said user analysis customization input, wherein said user selects and customizes one or more of said analysis customization options;
analyzing mapped input parameters, wherein said mapped input parameters are analyzed based on said analysis customization input;
generating one or more reports, wherein said one or more reports are based on the analysis of said mapped input parameters;
providing said user said one or more reports; wherein said one or more reports are selected from a group of reports comprising, charts, graphs, data tables, and text files.

8. The method of claim 6, further comprising the step of extracting said one or more reports to an external software program.

9. The method of claim 6, further comprising the step of storing said one or more reports for future use as said input parameters.
